(19) United States
(12) Patent Application Publication
(10) Pub. No.: US 2008/0009015 A1
(43) Pub. Date: Jan. 10, 2008

(54) Methods for Producing Olfactory Gpcrs

(75) Inventors: Gene Hung, San Diego, CA (US); Daniel Ortuno, Vista, CA (US); David Unett, San Diego, CA (US); Joel Gatlin, San Diego, CA (US)

Correspondence Address: BOZICEVIC, FIELD & FRANCIS LLP;(ARENA PHARMACEUTICALS, INC.), 1900 UNIVERSITY AVENUE, SUITE 200, EAST PALO ALTO, CA, 94303, US

(73) Assignee: Arena Pharmaceuticals, Inc., San Diego, CA (US)

(21) Appl. No.: 10/579,399

(22) PCT Filed: Nov. 15, 2004
(86) PCT No.: PCT/US04/38339

§ 371 Date: Mar. 12, 2007

Related U.S. Application Data
(60) Provisional application No. 60/523,940, filed on Nov. 21, 2003.

(51) Int. Cl. C07K 14/705; G01N 33/53; C12P 21/06

(52) U.S. Cl. 435/7.1; 435/368; 435/69.1; 530/350

(57) ABSTRACT

The subject invention provides a method for producing an olfactory GPCR in a cell. In general, the methods involve introducing an expression cassette containing a promoter operably linked to a nucleic acid encoding an olfactory PCR into a macroglial cell, e.g., a Schwann or oligodendritic cell, and maintaining the cell under conditions suitable for production of the olfactory GPCR. Also provided is a macroglial cell containing a recombinant nucleic acid encoding an olfactory GPCR, methods of screening for modulators of olfactory GPCR activity, and a kit for producing an olfactory GPCR in a macroglial cell. The invention finds most use in research on flavors and fragrances, and, consequently, has a variety of research and industrial applications.

This application claims the benefit of priority from the following provisional application, filed via U.S. Express mail with the United States Patent and Trademark Office on the indicated date: U.S. Provisional No. 60/523,940, filed Nov. 21, 2003. The disclosure of the foregoing application is herein incorporated by reference in its entirety.

### BACKGROUND OF THE INVENTION

1. Field of the Invention

The present invention relates to methods for producing GPCR proteins, particularly olfactory GPCR proteins, in a cell.

2. Background of the Invention

All animals possess a “nose”, an olfactory sense organ that allows for the recognition and discrimination of chemosensory information in the environment. Humans, for example, have a poor sense of smell compared to other animals, and yet they can perceive, i.e., smell, over 10,000 volatile chemicals (“odorants”) that are typically small organic molecules of less than 400 Da. These chemicals vary greatly in structure and include a panoply of diverse aliphatic acids, alcohols, aldehydes, ketones, and esters; chemicals with aromatic, alicyclic, polycyclic and heterocyclic ring structures; and innumerable substituted chemicals of each of these types, as well as combinations thereof. Remarkably, these molecules are not only detected by the olfactory system, they are discriminated by it.

Since some odors are desirable and other odors are repulsive, the ability to produce new odors, mimic odors, and manipulate perception of odor is extremely desirable. Towards this end, research into odor perception has intensified in recent years. In humans and other animal species, a large number of odorant receptors have been identified on olfactory cilia, a specialized type of dendrite of an olfactory sensory neuron. These odorant receptors exhibit a seven transmembrane domain topology characteristic of the superfamily of G-protein coupled receptors, and, accordingly, are termed “olfactory GPCRs”. Each olfactory sensory neuron expresses only one type of olfactory GPCR, and it is estimated that the human genome encodes approximately 500 active olfactory GPCRs.

Accordingly, a vast number of chemicals may be detected and discriminated by a relatively small number of receptors. This is achieved using a combinatorial receptor coding scheme in which each olfactory GPCR recognizes more than one odorant, each odorant is recognized by more than one olfactory GPCR. Thus odorants can be characterized according to the “fingerprint” of activated GPCRs. Once determined, this “fingerprint” provides the identity of the odorant as well as the basis for identification of other molecules that exhibit a similar “fingerprint” and thus smell.

Despite the fact that that recombinant olfactory GPCRs can be efficiently expressed at high levels by olfactory sensory neurons in vivo when exogenously introduced using an adenoviral vector (e.g., Touhara et al, Proc. Natl. Acad. Sci. 96: 4040-4045, 1999), olfactory GPCRs are quite exceptional in that they cannot be easily expressed in heterologous cultured cell systems in a manner that provides for their function in the cell (e.g., McClintock, Mol. Brain Res. 48:270-278, 1997). While the exact cause of this problem is not clear, one theory is that when expressed in non-endogenous cells, olfactory GPCRs are not exported to the plasma membrane of the cell and become sequestered in the endoplasmic reticulum. Another theory suggests that functional expression may be due to an olfactory specific factor required for proper membrane localization or inefficient coupling to transduction machinery in non-olfactory cells (Krieger et al, Eur. J. Biochem 219:829-835, 1994). Regardless of the mechanism that results in the inefficient and/or essentially non-functional expression of recombinant olfactory GPCRs in non-endogenous cultured cells, the solution to the problem has been previously unknown.

Until the present invention, olfactory GPCRs were exceptionally difficult to express in mammalian cells in vitro, and, even though such methods are extremely desirable, there were no robust, reliable and efficient method for producing and assaying those GPCRs. Progress in understanding odorant perception and discrimination has been severely hampered because olfactory GPCRs cannot be produced (Firestein Nature 413:211-218, 2001).

It follows from the foregoing that there is a great need for robust, reliable and efficient methods for producing olfactory GPCRs in a mammalian cell. This invention meets this need, and others, with unpredictably high level of success.

### LITERATURE

Literature of interest includes the following references: Zozulya et al, (Genome Biology 2:0018.1-0018.12, 2001; Mombairts (Annu. Rev. Neurosci 22:487-509, 1999); Raming et al, (Nature 361: 353-356, 1993); Belluscio et al, (Neuron 20: 69-81, 1988); Ronnet et al, (Annu. Rev. Physiol. 64:189-222, 2002); Lu et al, (Traffic 4: 416-533, 2003); Buck (Cell 100:611-618, 2000); Malnic et al, (Cell 96:713-723, 1999); Firestein (Nature 413:211-218, 2001); Zhao et al, (Science 279: 237-242, 1998); Touhara et al, (Proc. Natl. Acad. Sci. 96: 4040-4045, 1999); Sklar et al, (J. Biol. Chem 261:15538-15543, 1986); Dryer et al, (TiPS 20:413-417, 1999); Ivic et al, (J. Neurobiol. 50:56-68, 2002); and Fuchs et al, (Hum. Genet. 108:1-13, 2001); published US patent applications 20030143679 and 20030105285; and U.S. Pat. Nos. 6,610,511, 6,492,143 and 6,410,249.

### SUMMARY OF THE INVENTION

The subject invention provides a method for producing an olfactory GPCR in a cell. In general, the methods involve introducing an expression cassette containing a promoter operably linked to a nucleic acid encoding an olfactory GPCR into a macroglial cell, e.g., a Schwann or oligodendritic cell, and maintaining the cell under conditions suitable for production of the olfactory GPCR. Also provided is a macroglial cell containing a recombinant nucleic acid encoding an olfactory GPCR, methods of screening for modulators of olfactory GPCR activity, and a kit for producing an olfactory GPCR in a macroglial cell. The invention finds most use in research on flavors and fragrances, and, consequently, has a variety of research and industrial applications.

### BRIEF DESCRIPTION OF THE DRAWINGS

FIG. 1 is four panels of photographs showing expression of recombinant human olfactory GPCR on the surface of primary rat Schwann cells. Panel OR1 is olfactory GPCR having Genbank accession number P47893. Panel OR2 is olfactory GPCR having Genbank accession number NP_{—}036505. Panel OR3 is olfactory GPCR having Genbank accession number XP_{—}166868. Vector is empty expression vector negative control. Olfactory GPCR is expressed from a CMV promoter-based expression vector as an N-terminal fusion protein comprising a rhodopsin signal peptide and a hemagglutinin (HA) epitope tag.

### DEFINITIONS

Before the present invention is further described, it is to be understood that this invention is not limited to particular embodiments described, as such may of course vary. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this invention belongs.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, various publications, patents and published patent applications are cited. The disclosures of these publications, patents and published patent applications referenced in this application are hereby incorporated by reference in their entirety into the present disclosure. Citation herein by Applicant of a publication, patent, or published patent application is not an admission by Applicant of said publication, patent, or published patent application as prior art.

It must be noted that as used herein and in the appended claims, the singular forms “a”, “and”, and “the” include plural referents unless the context clearly dictates otherwise. Thus, for example, reference to “an agent” includes a plurality of such agents, and reference to “the GPCR” includes reference to one or more GPCRs and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

“G-protein coupled receptors”, or “GPCRs” are polypeptides that share a common structural motif, having seven regions of between 22 to 24 hydrophobic amino acids that form seven alpha helices, each of which spans a membrane [each span is identified by number, i.e., transmembrane-1 (TM1), transmembrane-2 (TM2), etc.]. The transmembrane helices are joined by regions of amino acids between transmembrane-2 and transmembrane-3, transmembrane-4 and transmembrane-5, and transmembrane-6 and transmembrane-7 on the exterior, or “extracellular” side, of the cell membrane [these are referred to as “extracellular” regions 1, 2 and 3 (EC1, EC2 and EC3), respectively]. The transmembrane helices are also joined by regions of amino acids between transmembrane-1 and transmembrane-2, transmembrane-3 and transmembrane-4, and transmembrane-5 and transmembrane-6 on the interior, or “intracellular” side, of the cell membrane [these are referred to as “intracellular” regions 1, 2 and 3 (IC1, IC2 and IC3), respectively]. The “carboxy” (“C”) terminus of the receptor lies in the intracellular space within the cell, and the “amino” (“N”) terminus of the receptor lies in the extracellular space outside of the cell. GPCR structure and classification is generally well known in the art, and further discussion of GPCRs may be found in Probst, DNA Cell Biol. 1992 11:1-20; Marchese et al Genomics 23: 609-618, 1994; and the following books: Jürgen Wess (Ed) Structure-Function Analysis of G Protein-Coupled Receptors published by Wiley-Liss (1st edition; Oct. 15, 1999); Kevin R. Lynch (Ed) Identification and Expression of G Protein-Coupled Receptors published by John Wiley & Sons (March 1998) and Tatsuya Haga (Ed), G Protein-Coupled Receptors, published by CRC Press (Sep. 24, 1999); and Steve Watson (Ed) G-Protein Linked Receptor Factsbook, published by Academic Press (1st edition; 1994).

A “native GPCR” is a GPCR that is produced by an animal, e.g., a mammal such as a human or mouse. Detailed description of native GPCRs may be found in the On-line Mendelian Inheritance in Man database found at the world wide website of the National Center of Biotechnology Information (NCBI). Additional description of native GPCRs may be found at the world wide website of primalinc.com and a list of exemplary GPCRs for use in the subject methods is set forth in Table 1.

The term “ligand” means a molecule that specifically binds to a GPCR A ligand may be, for example a polypeptide, a lipid, a small molecule or an antibody, etc. A “native ligand” is a ligand that is an endogenous, natural ligand for a native GPCR. A ligand may be a GPCR “antagonist”, “agonist”, “partial agonist” or “inverse agonist”, or the like.

A “modulator” is a ligand that increases or decreases a GPCR intracellular response when it is in contact with, e.g., binds, to a GPCR that is expressed in a cell.

The term “second messenger” shall mean an intracellular response produced as a result of receptor activation. A second messenger can include, for example, inositol 1,4,5-triphosphate (IP3), diacylglycerol (DAG), cyclic AMP (cAMP), cyclic GMP (cGMP), and Ca2+. Second messenger response can be measured for a determination of receptor activation. In addition, second messenger response can be measured for the identification of candidate agents as, for example, agonists, partial agonists, inverse agonists, and antagonists.

An “agonist” is a ligand which activates a GPCR intracellular response when it binds to a GPCR.

A “partial agonist” is a ligand what activates, to a lesser extent than an agonist, a GPCR intracellular response when it binds to a GPCR.

An “antagonist” is a ligand which competitively binds to a GPCR at the same site as an agonist but which does not activate the intracellular response produced by the active form of a GPCR. Antagonists usually inhibit intracellular responses by an agonist or partial agonist. Antagonists usually do not diminish the baseline intracellular response in the absence of an agonist or partial agonist.

An “inverse agonist” is a ligand which binds to a GPCR and inhibits the baseline (basal) intracellular response of the GPCR observed in the absence of an agonist or partial agonist. In most embodiments, a baseline intracellular response is inhibited in the presence of an inverse agonist by at least about 30%, by at least about 50%, or by at least 75%, as compared to a baseline response in the absence of an inverse agonist.

The term “odorant” encompasses any compound, naturally occurring or chemically synthesized, of known or unknown structure, that activates an olfactory GPCR. As discussed in the Background section above, odorants are usually volatile, small organic molecules of less than 400 Da. Flavors, perfumes, scents, odors, fragrance are types of odorants. An “odor” is the sensation associated with a particular odorant.

The term “phenomenon associated with olfactory GPCR activity” as used herein refers to a structural, molecular, or functional characteristic associated with olfactory GPCR activity, particularly such a characteristic that is readily assessable in a human or animal model. Such characteristics include, but are not limited to, downstream molecular events caused by activation of a GPCR, and sensory phenotypes such as smell, taste, or other behavioral or physiological events caused by activation of a GPCR.

A “deletion” is defined as a change in either amino acid or nucleotide sequence in which one or more amino acid or nucleotide residues, respectively, are absent as compared to an amino acid sequence or nucleotide sequence of a parental GPCR polypeptide or nucleic acid. In the context of a GPCR or a fragment thereof, a deletion can involve deletion of about 2, about 5, about 10, up to about 20, up to about 30 or up to about 50 or more amino acids. A GPCR or a fragment thereof may contain more than one deletion.

An “insertion” or “addition” is that change in an amino acid or nucleotide sequence which has resulted in the addition of one or more amino acid or nucleotide residues, respectively, as compared to an amino acid sequence or nucleotide sequence of a parental GPCR. “Insertion” generally refers to addition to one or more amino acid residues within an amino acid sequence of a polypeptide, while “addition” can be an insertion or refer to amino acid residues added at an N- or C-terminus, or both termini. In the context of a GPCR or fragment thereof, an insertion or addition is usually of about 1, about 3, about 5, about 10, up to about 20, up to about 30 or up to about 50 or more amino acids. A GPCR or fragment thereof may contain more than one insertion.

A “substitution” results from the replacement of one or more amino acids or nucleotides by different amino acids or nucleotides, respectively as compared to an amino acid sequence or nucleotide sequence of a parental GPCR or a fragement thereof. It is understood that a GPCR or a fragment thereof may have conservative amino acid substitutions which have substantially no effect on GPCR activity. By conservative substitutions is intended combinations such as gly, ala; val, ile, leu; asp, glu; asn, gln; ser, thr; lys, arg; and phe, tyr.

The term “biologically active” GPCR refers to a GPCR having structural and biochemical functions of a naturally occurring GPCR.

As used herein, the terms “determining,” “measuring,” “assessing,” and “assaying” are used interchangeably and include both quantitative and qualitative determinations. Reference to an “amount” of a GPCR in these contexts is not intended to require quantitative assessment, and may be either qualitative or quantitative, unless specifically indicated otherwise.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially pure” refers to a compound that is removed from its natural environment and is at least 60% free, preferably 75% free, and most preferably 90% free from other components with which it is naturally associated.

A “coding sequence” or a sequence that “encodes” a selected polypeptide, is a nucleic acid molecule which can be transcribed (in the case of DNA) and translated (in the case of mRNA) into a polypeptide, for example, in a host cell when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or prokaryotic DNA, and synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences that are immunologically identifiable with a polypeptide encoded by the sequence.

“Operably linked” refers to an arrangement of elements wherein the components so described are configured so as to perform their usual function. A promoter that is operably linked to a coding sequence will effect the expression of a coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

A “vector” is capable of transferring gene sequences to a host cell. Typically, “vector construct,” “expression vector,” and “gene transfer vector,” mean any nucleic acid construct capable of directing the expression of a gene of interest and which can transfer gene sequences to host cells, which can be accomplished by genomic integration of all or a portion of the vector, or transient or inheritable maintenance of the vector as an extrachromosomal element. Thus, the term includes cloning, and expression vehicles, as well as integrating vectors.

An “expression cassette” comprises any nucleic acid construct capable of directing the expression of a gene/coding sequence of interest, which is operably linked to a promoter of the expression cassette. Such cassettes can be constructed into a “vector,” “vector construct,” “expression vector,” or “gene transfer vector,” in order to transfer the expression cassette into a host cell. Thus, the term includes cloning and expression vehicles, as well as viral vectors.

A first polynucleotide is “derived from” or “corresponds to” a second polynucleotide if it has the same or substantially the same nucleotide sequence as a region of the second polynucleotide, its cDNA, complements thereof, or if it displays sequence identity as described above.

A first polypeptide is “derived from” or “corresponds to” a second polypeptide if it is (i) encoded by a first polynucleotide derived from a second polynucleotide, or (ii) displays sequence identity to the second polypeptides as described above.

The terms “administering”, and the like, refers to adding a GPCR modulatory agent to obtain a desired pharmacologic and/or physiologic effect. In many embodiments, the subject GPCR modulatory agents are volatile, and, as such, they are administered orally or intranasally, either directly or indirectly by addition to foodstuffs or to the atmosphere. The effect may completely or partially prevent perception of an odor, may increase perception of an odorant, or may generate a new odor.

The term “non-naturally occurring” or “recombinant” means artificial or otherwise not found in nature. Recombinant cells usually contain nucleic acid that is not usually found in that cell, recombinant nucleic acid usually contain a fusion of two or more nucleic acids that is not found in nature, and a recombinant polypeptide is usually produced by a recombinant nucleic acid.

“Subject”, “individual,” “host” and “patient” are used interchangeably herein, to refer to any animal, e.g., mammal, human or non-human, having olfactory GPCRs. Generally, the subject is a mammalian subject. Exemplary subjects include, but are not necessarily limited to, humans, non-human primates, mice, rats, cattle, sheep, goats, pigs, dogs, cats, and horses, with humans being of particular interest.

### DETAILED DESCRIPTION OF THE INVENTION

The subject invention provides a method for producing an olfactory GPCR in a cell. In general, the methods involve introducing an expression cassette containing a promoter operably linked to a nucleic acid encoding an olfactory GPCR into a macroglial cell, e.g., a Schwann or oligodendritic cell, and maintaining the cell under conditions suitable for production of the olfactory GPCR. Also provided is a macroglial cell containing a recombinant nucleic acid encoding an olfactory GPCR, methods of screening for modulators of olfactory GPCR activity, and a kit for producing an olfactory GPCR in a macroglial cell. The invention finds use in, for example, analysis and identification of flavors and fragrances, and, consequently, has a variety of research and industrial applications.

In further describing the invention in greater detail than provided in the Summary and as informed by the Background and Definitions provided above, methods of producing an olfactory GPCR are described first, followed by a description of compositions and kits that find use in performing the subject methods. Finally, methods of screening for modulators of olfactory GPCR activity and methods of screening for odorant mimetics are discussed.

Methods for Producing an Olfactory GPCR

In one aspect, the invention provides methods of producing an olfactory GPCR in a cell. In describing these methods, the compositions for use in the methods will be described first.

Olfactory G-Protein Coupled Receptors

The term “olfactory G-protein coupled receptor” (or abbreviations thereof, e.g., “olfactory GPCR”) refers to any member of a phylogenetically distinct, art-recognized sub-family of the GPCR superfamily that is involved in chemosensation. Olfactory GPCRs are both generally and specifically disclosed in a wide variety of publications and public databases, including Zozulya et al, (Genome Biol. 2:0018, 2001); Glusman et al, (Genome Res. 11: 685-702, 2001) and Crasto et al, (Nucleic Acids Res. 30:354-60, 2002), which are specifically incorporated herein in their entirety. In particular, the olfactory GPCRs set forth in the database of olfactory GPCR sequences found at the world wide website of the Senselab.med.yale.edu are of interest. A non-limiting list of exemplary olfactory GPCRs suitable for use in the subject methods is provided in Table 1, inserted before the claims. Table 1 is a list of accession numbers of protein sequence entries from the Swiss-Prot database, as found at the world wide website of the European Bioinformatics Institute. These database entries listed in Table 1, in particular the amino acids sequences set forth in those entries, are specifically incorporated herein by reference in their entirety.

It is expressly contemplated that the olfactory GPCR may be of human origin or of non-human animal origin. In certain embodiments, the non-human animal may be a mouse, a rat, a dog, or any other non-human animal with an acute and discriminating sense of smell. In certain embodiments, the olfactory GPCR may be of insect origin (e.g., mosquito, ant, aphid, beetle, fly, wasp, bee, spider, or any insect which transmits a disease to human or non-human animals or which causes damage to crops or ornamental plants). In particular embodiments, the olfactory GPCR is human.

It is recognized that both native and altered native olfactory GPCRs may be used in the subject methods. Accordingly, the term “olfactory G-protein coupled receptor” is also intended to encompass an altered native olfactory GPCR (e.g. a native olfactory GPCR that is altered by addition such as an addition of a reporter, substitution, deletions and insertions, etc.) such that it binds the same ligand as a corresponding native GPCR.

The term “olfactory G-protein coupled receptor” therefore includes variants of the GPCR polypeptides recited in Table 1. In other words, variants of any olfactory GPCR may be used in the subject methods. In certain embodiments, therefore, an olfactory GPCR may have an altered sequence as compared to a native sequence (e.g., a sequence deposited in NCBI's Genbank database or the like). For example, an olfactory GPCR may be a native polypeptide having any number of amino acid substitutions, amino acid deletions, or amino acid additions at any position in the polypeptide (e.g., the C- or N-terminus, or at internal positions).

In particular embodiments, the olfactory GPCR is a fusion protein, and may contain, for example, an affinity tag domain or a reporter domain. Suitable affinity tags include any amino acid sequence that may be specifically bound to another moiety, usually another polypeptide, most usually an antibody. Suitable affinity tags include epitope tags, for example, the V5 tag, the FLAG tag, the HA tag (from hemagglutinin influenza virus), the myc tag, and the like, as is known in the art. Suitable affinity tags also include domains for which, binding substrates are known, e.g., HIS, GST and MBP tags, as is known in the art, and domains from other proteins for which specific binding partners, e.g., antibodies, particularly monoclonal antibodies, are available. Suitable affinity tags also include any protein-protein interaction domain, such as a IgG Fc region, which may be specifically bound and detected using a suitable binding partner, e.g. the IgG Fc receptor. It is expressly contemplated that such a fusion protein may contain a heterologous N-terminal domain (e.g. an epitope tag) fused in-frame with a GPCR that has had its N-terminal methionine residue either deleted or substituted with an alternative amino acid. In certain embodiments, the olfactory GPCR fusion protein may comprise at its N-terminus a rhodopsin signal peptide alone or in combination with a hemagglutinin epitope tag. In particular embodiments, the olfactory GPCR fusion protein may comprise an N-terminus having the amino acid sequence MNGTEGPNFYVPFSNKTGVVYPYDVPDYAKL,

where MNGTEGPNFYVPFSNKTGVV is rhodopsin signal peptide and YPYDVPDYAKL is hemagglutinin epitope tag. It is well within the purview of persons of skill in the art to construct an expression cassette allowing for the expression of the olfactory GPCR as a fusion protein (see, e.g., Krautwurst et al, Cell 95:917-926, 1998). It is appreciated that a polypeptide of interest may first be made from a native polypeptide and then operably linked to a suitable reporter/tag as described above. In other embodiments, an olfactory GPCR may be a fragment of a GPCR, wherein said GPCR fragment is biologically active.

Suitable reporter domains include any domain that can report the presence of a polypeptide. While it is recognized that an affinity tag may be used to report the presence of a polypeptide using, e.g., a labeled antibody that specifically binds to the tag, light emitting reporter domains are more usually used. Suitable light emitting reporter domains include luciferase (from, e.g., firefly, Vargula, Renilla reniformis or Renilla muelleri), and light emitting variants thereof. Other suitable reporter domains include fluorescent proteins (from e.g., jellyfish, corals and other coelenterates as such those from Aequoria, Renilla, Ptilosarcus, Stylatula species), or light emitting variants thereof. Light emitting variants of these reporter proteins are very well known in the art and may be brighter, dimmer, or have different excitation and/or emission spectra, as compared to a native reporter protein. For example, some variants are altered such that they no longer appear green, and may appear blue, cyan, yellow, enhanced yellow red (termed BFP, CFP, YFP eYFP and RFP, respectively) or have other emission spectra, as is known in the art. Other suitable reporter domains include domains that can report the presence of a polypeptide through a biochemical or color change, such as β-galactosidase, β-glucuronidase, chloramphenicol acetyl transferase, and secreted embryonic alkaline phosphatase. In some preferred embodiments, the reporter domain is Renilla luciferase (e.g., pRLCMV; Promega, catalog number E2661).

Also, as is known in the art, an affinity tags or a reporter domain may be present at any position in an olfactory GPCR. However, in most embodiments, they are present at the C- or N-terminal end of an olfactory GPCR.

In many embodiments, an olfactory GPCR is a member of a library of olfactory GPCRs. Typically, a library contains a plurality of members, where a plurality may be 2 or more, 5 or more, about 10 or more, about 20 or more, about 50 or more, about 100 or more, about 200 or more, about 300 or more, about 500 or more, about 1000 or more, or even up to about 10,000 or more. The library may therefore contain about 5, about 10, about 20, about 30 or more, about 50 or more, about 100 or more, about 200 or more, usually up to 500 or more, usually up to about 1000 or more olfactory GPCR polypeptides. The members of the library may be of known identity, or unknown identity, or a mixture thereof. The members of the library may be entirely derived from one species or may be derived from a plurality of species.

Nucleic Acids Encoding Olfactory G-Protein Coupled Receptors

Since the genetic code and recombinant techniques for manipulating nucleic acid are known, and the amino acid sequences of olfactory GPCR polypeptides are described above, the design and production of nucleic acids encoding an olfactory GPCR polypeptide is well within the skill of an artisan. In certain embodiments, standard recombinant DNA technology (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) methods are used. For example, olfactory GPCR coding sequences may be isolated from a library of olfactory GPCR coding sequence using any one or a combination of a variety of recombinant methods that do not need to be described herein in any great detail. Subsequent substitution, deletion, and/or addition of nucleotides in the nucleic acid sequence encoding a protein may also be done using standard recombinant DNA techniques.

For example, site directed mutagenesis and subcloning may be used to introduce/delete/substitute nucleic acid residues in a polynucleotide encoding a polypeptide of interest. In other embodiments, PCR may be used. Nucleic acids encoding a polypeptide of interest may also be made by chemical synthesis entirely from oligonucleotides (e.g., Cello et al., Science (2002) 297:1016-8).

In certain embodiments, the codons of the nucleic acids encoding polypeptides of interest are optimized for expression in cells of a particular species, particularly a mammalian, e.g., human or mouse species.

The invention further provides vectors (also referred to as “constructs”) comprising a subject nucleic acid. In many embodiments of the invention, the subject nucleic acid sequences will be expressed in a host after the sequences have been operably linked to an expression control sequence, including, e.g. a promoter to form an expression cassette. A subject expression cassette is typically placed in an expression vector that can replicate in a host cell either as an episome or as an integral part of the host chromosomal DNA. Commonly, expression vectors will contain selection markers, e.g., tetracycline or neomycin, to permit detection of those cells transformed with the desired DNA sequences (see, e.g., U.S. Pat. No. 4,704,362, which is incorporated herein by reference). Vectors, including single and dual expression cassette vectors are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Suitable vectors include viral vectors, plasmids, cosmids, artificial chromosomes human artificial chromosomes, bacterial artificial chromosomes, yeast artificial chromosomes, etc.), mini-chromosomes, and the like. Retroviral, adenoviral and adeno-associated viral vectors may be used.

A variety of expression vectors are available to those in the art for purposes of producing a polypeptide of interest in a cell. One suitable vector is pCMV, which used in certain embodiments. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be viable. The ATCC has assigned the following deposit number to pCMV: ATCC #203351.

The subject expression cassettes usually comprise a single open reading frame encoding an olfactory GPCR, however, in certain embodiments, since the host cell for expression of the olfactory GPCR may be a eukaryotic cell, e.g., a mammalian cell, such as a human cell, the open reading frame may be interrupted by introns. Subject expression cassettes are typically part of a transcriptional unit which may contain, in addition to the subject nucleic acid 3′ and 5′ untranslated regions (UTRs) which may direct RNA stability, translational efficiency, etc. The expression cassette may also be part of an nucleic acid which contains, in addition to the subject nucleic acid, a transcriptional terminator.

The subject expression cassettes may comprise nucleic acid sequence allowing for expression of the olfactory GPCR as a fusion protein. In certain embodiments, the olfactory GPCR fusion protein may comprise at its N-terminus a rhodopsin signal peptide and/or a hemagglutinin epitope tag. In particular embodiments, the olfactory GPCR fusion protein may comprise an N-terminus having the amino acid sequence MNGTEGPNFYVPFSNKTGVVYPYDVPDYAKL,

where MNGTEGPNFYVPFSNKTGVV is rhodopsin signal peptide and YPYDVPDYAKL is hemagglutinin epitope tag. It is well within the purview of persons of skill in the art to construct an expression cassette allowing for the expression of the olfactory GPCR as a fusion protein (see, e.g., Krautwurst et al, Cell 95:917-926, 1998).

Eukaryotic promoters (i.e., promoters that function in a eukaryotic cell) can be any promoter that is functional in a macroglial cell, including viral promoters and promoters derived from eukaryotic genes. Exemplary eukaryotic promoters include, but are not limited to, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982; Silver et al., Proc. Natl. Acad. Sci. (USA) 81:5951-59SS, 1984), the CMV promoter, the EF-1 promoter, Ecdysone-responsive promoter(s), tetracycline-responsive promoter, and the like. Viral promoters may be of particular interest as they are generally particularly strong promoters. In certain embodiments, a promoter is used that is a viral promoter. Promoters for use in the present invention are selected such that they are functional in the macroglial cells (and/or animal) into which they are being introduced. In certain embodiments, the promoter is a CMV promoter.

In certain embodiments, a subject vector may also provide for expression of a selectable marker. Suitable vectors and selectable markers are well known in the art and discussed in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). A variety of different genes have been employed as selectable markers, and the particular gene employed in the subject vectors as a selectable marker is chosen primarily as a matter of convenience. Known selectable marker genes include: the thimydine kinase gene, the dihydrofolate reductase gene, the xanthine-guanine phosphoribosyl transferase gene, CAD, the adenosine deaminase gene, the asparagine synthetase gene, the antibiotic resistance genes, e.g. tetr, ampr, Cmr or cat, kanr or neor (aminoglycoside phosphotransferase genes), the hygromycin B phosphotransferase gene, and the like.

As mentioned above, olfactory GPCRs may be fusion proteins that contain an affinity domain and/or a reporter domain. Methods for making fusions between a reporter or tag and a GPCR, for example, at the C- or N-terminus of the GPCR, are well within the skill of one of skill in the art (e.g. McLean et al, Mol. Pharma. Mol Pharmacol. 1999 56:1182-91; Ramsay et al., Br. J. Pharmacology, 2001, 315-323) and will not be described any further. It is expressly contemplated that such a fusion protein may contain a heterologous N-terminal domain (e.g., an epitope tag) fused in-frame with a GPCR that has had its N-terminal methionine residue either deleted or substituted with an alternative amino acid. It is appreciated that a polypeptide of interest may first be made from a native polypeptide and then operably linked to a suitable reporter/tag as described above.

The subject nucleic acids may also contain restriction sites, multiple cloning sites, primer binding sites, ligatable ends recombination sites etc., usually in order to facilitate the construction of a nucleic acid encoding an olfactory GPCR.

Since an olfactory GPCR may be member of a library of polypeptides of interest, the nucleic acids encoding such a polypeptide of interest may also be a similar sized library of nucleic acids encoding olfactory GPCRs.

Host Cells

The methods described herein generally involve producing an olfactory GPCR in a cultured macroglial cell (i.e., a primary or immortal macroglial cell cultured in vitro). By “macroglial cell” is meant any cell of a variety of neuron-associated cell types, including:

Schwann cells, oligodendrocytes and astrocytes, and derivatives thereof. In many embodiments, suitable host cells may be “myelin-producing” cells that produce myelin, the material that forms sheath of nerve axons. Myelin-producing macroglial cells include Schwann cells, oligodendrocytes, as well as certain types of astrocytes that produce myelin (e.g., olfactory sheathing cells). Myelin producing cells can usually be identified by their synthesis of a galactocerebroside, gal C, which is a component of myelin.

Also encompassed by the term “macroglial cells” are modified versions of macroglial cells, including cancerous macroglial cells, e.g., Schwanoma, neurofibromas, astrocytoma cells, and oligodendrocytoma cells; immortal macroglial cells, e.g., cells immortalized via introduction of a suitable oncogenes, e.g., HPV E6-E7, T antigen, and the like; hybrid cells produced by cell fusion in which a macroglial cell is fused with a different (non-macroglial) or a like (macroglial) type of cell; and recombinant macroglial cells, e.g., cells that have contain an exogenous nucleic acid, or a “knockout” in an endogenous gene, e.g., a gene required for or that inhibits the synthesis of myelin. Macroglial cells are usually from mammalian species, such as rodents (e.g., mouse) or humans. Exemplary and non-limiting cell lines include RN2 and EJ (Coulter-Mackie, Virus Research 1:477-487, 1984), RN22 (Kreider, Brain Research 397:238-244, 1986), and HOG and MO3.13 (Buntinx, Journal of Neurocytology 32:25-38, 2003). A macroglial cell recombinant for other than an olfactory GPCR is expressly contemplated to be encompassed by the term “macroglial cell.”

Accordingly, since methods of culturing macroglial cells are well known in the art, (see, e.g., Mosahebi Glia, 34:8-17, 2001; Shen, Microsurgery 19:356-63, 1999; Acta Neuropathol (Berl), 78:317-24, 1989; Barnett, Developmental Biology, 155: 337-350, 1993; and Hung et al, International Journal of Oncology 20: 475-482, 2002) a variety of suitable host cells are available for production of olfactory GPCRs, including immortalized HEI193 cells and the like.

In particular, Schwann cells may be cultured using the following methods: Hung, (Int. J. Oncol. 20:475-82, 2002); Hung, (Int. J. Oncol. 1999 14:409-15); Wood, (Brain Res. 115:361-75, 1976); Wood, (Ann. N.Y. Acad. Sci. 605:1-14, 1990); and Brockes, (J. Exp. Biol. December; 95:215-30, 1981).

Additional cell lines will become apparent to those of ordinary skill in the art, and are available from the American Type Culture Collection, 10801 University Boulevard, Manassas, Va. 20110-2209.

Methods

In general accordance with the subject methods, an olfactory GPCR expression cassette is introduced into a macroglial cell in vitro, the cell is subjected to conditions suitable for expression of the olfactory GPCR, and the GPCR is expressed in the cell and exported to the cell surface.

Accordingly, in most embodiments, an expression cassette may be introduced into a host cell using a variety of methods, including viral infection, transfection, conjugation, protoplast fusion, electroporation, calcium phosphate precipitation, direct microinjection, and the like. The choice of method is generally dependent on the type of cell being transformed and the circumstances under which the transformation is taking place (e.g., in vitro, etc.). A general discussion of these methods can be found in Ausubel, et al, Short Protocols in Molecular Biology, 3rd ed., Wiley & Sons, 1995.

After introduction of an expression cassette for an olfactory GPCR into a cell, the cell is typically incubated to provide for polypeptide expression. To accomplish this, the cell may be incubated in suitable media for 12-24 hr, 24-48 hr, or 48-96 hr or more. Transient expression of the polypeptide may be carried out in this manner. It is expressly contemplated, however, that expression of the polypeptide may alternatively be stable. In stable transfection, the expression cassette contains a selectable marker gene and establishment of a stable cell line expressing the polypeptide involves selection for the selectable marker gene. If two expression cassettes are introduced into a cell, the two expression cassettes usually contain two different selectable marker genes (e.g., neomycin resistance gene and hygromycin resistance gene). Methods of transient and stable transfection are well known to those of skill in the art.

Olfactory GPCRs are produced in the macroglial cell, and usually exported to the surface of the cell such that the GPCR is present in the plasma membrane.

Compositions

In another aspect, the invention provides a macroglial cell producing a biologically active olfactory GPCR. Such cells usually contain a recombinant nucleic acid encoding an olfactory GPCR, and may produce an olfactory GPCR that is not usually produced in that cell (i.e. a macroglial cell in the absence of the recombinant nucleic acid).

As mentioned above, the present invention provides a macroglial cell containing an olfactory GPCR that is present (i.e., detectably present) at the surface of the macroglial cell, usually spanning the plasma membrane of the cell in a manner that is characteristic of GPCRs. Accordingly, the subject cells contain “active” olfactory GPCRs in that they are capable of binding a ligand, and transmitting a signal via a suitable G-protein, if present. The subject cells thus find use in activity assays, e.g., screening assays, which will be described in great detail below.

The subject cells usually produce olfactory GPCR at a significantly level greater than that of control cells such as a non-macroglial cells, e.g. an NIH-3T3 cell, COS cell, or the like, into which the same expression cassette has been produced. In most embodiments, the subject cells produce, on a molar basis, at least 5× (“5 times”), at least 10×, at least 50×, at least 100×, usually up to at least 1000× more olfactory GPCR than control cells. In particular embodiments, the subject cells produce, on a molar basis, at least 5× (“5 times”), at least 10×, at least 50×, at least 100×, usually up to at least 1000× more olfactory GPCR at the cell surface than control cells (e.g., as determined by immunocytochemistry or flow cytometry). When the subject cells are grown in liquid culture, they usually produce olfactory GPCR in significant amounts, e.g., greater than 10 μg/l, greater than 100 μg/l, greater than 1 mg/l, greater than 10 mg/l or greater than about 50 mg/l or more. In particular, when the subject cells are grown in liquid culture, they usually produce cell surface olfactory GPCR in significant amounts, e.g., greater than 10 μg/l, greater than 100 μg/l, greater than 1 mg/l, greater than 10 mg/l or greater than about 50 mg/l or more.

Since there are a number of different olfactory GPCRs, the invention also provides a plurality of macroglial cells (i.e., a library of macroglial cells) containing a corresponding plurality of recombinant nucleic acids encoding different olfactory GPCRs. In these embodiments, each macroglial cell of the plurality usually contains a recombinant nucleic acid for a single olfactory GPCR, and each cell contains a different nucleic acid. Accordingly, the invention provides a library of macroglial cells, the cells containing recombinant nucleic acids encoding 2 or more, 5 or more, about 10 or more, about 20 or more, about 50 or more, about 100 or more, about 200 or more, about 300 or more, about 500 or more, about 1000 or more different olfactory GPCRs. The olfactory GPCRs may be of known identity, or unknown identity, or a mixture thereof. The olfactory GPCRs may be derived from a single species or alternatively derived from 2, up to about 5, up to about 10, up to about 50, up to about 100, or up to about 1000 species of animal. In certain embodiments, the olfactory GPCRs are human.

Kits

Also provided by the subject invention are kits for practicing the subject methods, as described above. The subject kits at least include one or more of: a macroglial cell, a nucleic acid encoding an olfactory GPCR, and a macroglial cell containing an olfactory GPCR. The nucleic acids of the kit may also have restrictions sites, multiple cloning sites, primer sites, etc to facilitate their ligation into other plasmids. Other optional components of the kit include: culture media, components for testing GPCR activity, and G-protein-encoding nucleic acids, etc, for performing the subject assays. The various components of the kit may be present in separate containers or certain compatible components may be precombined into a single container, as desired.

In addition to above-mentioned components, the subject kits typically further include instructions for using the components of the kit to practice the subject methods, e.g., methods of producing an olfactory GPCR, etc. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Methods for Identifying Modulators of Olfactory GPCR Activity

The invention provides methods of screening for olfactory GPCR modulators (i.e., compounds that increase or decrease the activity of an olfactory GPCR of interest). In certain embodiments, the olfactory GPCR modulator is selected from the group consisting of agonist, partial agonist, inverse agonist, and antagonist. In general, the methods involve producing an olfactory GPCR in a macroglial cell according to the methods described above to provide a macroglial cell producing a biologically active olfactory GPCR (termed herein a “subject macroglial cell”), contacting the cell with a candidate agent, and assessing the effect of the candidate agent on an activity of the olfactory GPCR.

In other embodiments, a modulator of an olfactory GPCR (e.g. a natural or synthetic ligand for that GPCR, for example) may be contacted with a macroglial producing that GPCR, and the effect of the modulator on the activity of the olfactory GPCR may be assessed. Also envisioned are assays that are done using two olfactory GPCR modulators, e.g., an activator of an olfactory GPCR (for example, a ligand for that GPCR), and an agent that blocks the modulatory activity of the activator.

As is known in the art, the subject assays may be performed using a variety of methods, such as, for example, membrane binding assays using ³⁵S GTPγS, adenylyl cyclase assays (e.g., using the FLASH PLATE™ Adenylyl Cyclase kit from New England Nuclear; Cat. No. SMP004A), cell-based cAMP assays, reporter-based assays, AP1 reporter assays, SRF-Luc reporter assays, intracellular IP3 accumulation assays, fluorometric imaging plate reader (FLIPR) assays for the measurement of intracellular calcium concentration, and the like.

In embodiments where the modulator increases olfactory GPCR activity, the activity of an olfactory GPCR is increased in the presence of the modulator by at least about 10%, by at least about 20%, by at least about 30%, by at least about 50%, by at least about 80%, by at least about 100%, by at least about 500%, or by at least about 10-fold or more, as compared to suitable controls in the absence of the agent. Suitable controls may be in the presence or absence of the native ligand for the GPCR.

In embodiments where the modulator decreases olfactory GPCR activity, the activity of the olfactory GPCR is decreases in the presence of the modulator by at least about 10%, by at least about 20%, by at least about 30%, by at least about 50%, by at least about 70%, by at least about 80%; by at least about 90%, or by at least about 95% or more, as compared to suitable controls in the absence of the agent. Suitable controls may be in the presence or absence of the native ligand for the GPCR.

In certain embodiments, these methods also involve measuring GPCR activity in the presence or absence of a test compound, e.g., a candidate agent. These assays may involve contacting an isolated subject macroglial cell (e.g., a cultured cell), a membrane isolated from a subject macroglial cell, an extract of a subject macroglial cell, with an amount of a GPCR modulator that is effective to modulate the activity of the GPCR.

Accordingly, the invention provides for inhibitors of olfactory GPCR activity to reduce the activity of an olfactory GPCR in the presence or absence of a ligand, e.g., a natural ligand, for that GPCR, and inducers of GPCR activity, where the GPCR is induced by a compound that is or is not the natural ligand of the GPCR.

In certain embodiments, GPCR activity may be measured by assessing a reporter signal. In these embodiments, the assays may be performed in a format suitable for high throughput assays, e.g., 96- or 384-well format, and suitable robots, (e.g., pipetting robots), and instrumentation (96- or 384-well format luminometers or fluorescence readers for determining reporter activity) may be used. By way of illustration and not limitation, determining reporter activity may employ a Wallac 1450 Microbeta counter (Perkin-Elmer) or a CCD camera-based illuminator.

In related embodiments, the assay may be a binding assay, wherein the binding of a candidate agent to an olfactory GPCR is assessed. In these embodiments, the candidate agent is usually first labeled, contacted with a subject macroglial cell, and binding of the agent to the macroglial cell assessed.

Candidate Agents

A variety of different test compounds may be screened by the above methods. Test compounds encompass numerous chemical classes, though typically they are organic molecules, preferably small organic compounds (i.e., compounds having a molecular weight of more than 50 and less than about 2,500 daltons (e.g, 100-1000 Da, usually less than about 500 Da)). Test compounds comprise functional groups necessary for structural interaction with proteins, particularly hydrogen bonding, and typically include at least an amine, carbonyl, hydroxyl or carboxyl group, preferably at least two of the functional chemical groups. The test compounds often comprise cyclical carbon or heterocyclic structures and/or aromatic or polyaromatic structures substituted with one or more of the above functional groups. Exemplary and non-limiting test compounds include aliphatic acids, alcohols, ketones, and esters; chemicals with aromatic, alicyclic, polycyclic and heterocyclic ring structures; and innumerable substituted chemicals of each of these types, as well as combinations thereof. Test compounds are also found among biomolecules including peptides, saccharides, fatty acids, steroids, purines, pyrimidines, derivatives, structural analogs or combinations thereof. Further test compounds include variants of a GCPR's native ligand.

Test compounds may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. A library may preferentially comprise natural or synthetically produced compounds associated with smell. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Of interest are test compounds that are polypeptides, e.g., proteinaceous, agents. A specific type of polypeptide test compound of interest is an antibody for the GPCR, or a GPCR-binding fragment thereof. The antibody may be monoclonal or polyclonal, and may be produced according to methods known in the art. Further test compounds include variants of the GCPR's native ligand, for a GPCR having a known native ligand, e.g. a native ligand that is altered by substitution, deletion or addition of at least one amino acid, or chemically modified. In certain embodiments test compounds include endogenous polypeptides not known to be ligands of the GPCR.

The foregoing characterization of test compounds is intended to be illustrative and not limiting.

Methods for Identifying a Candidate Agent as a Ligand of an Olfactory GPCR.

A ligand of an olfactory GPCR may be identified by contacting a candidate agent with the olfactory GPCR and determining whether the candidate agent binds to the olfactory GPCR, wherein said binding is indicative of the candidate agent being a ligand of the olfactory GPCR. In certain embodiments, the candidate agent may be labeled. In particular embodiments, the candidate agent may be radiolabeled.

Suitable radionuclides that may be incorporated into subject candidate agents include but are not limited to ²H (deuterium), ³H (tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. Incorporation of ³H, ¹⁴C, ⁸²Br, ¹²⁵I, ¹³¹I, ³⁵S or may generally be most useful.

Synthetic methods for incorporating radio-isotopes into organic compounds are applicable to subject candidate agents and are well known in the art. These synthetic methods, for example, incorporating activity levels of tritium into target molecules, are as follows:

A. Catalytic Reduction with Tritium Gas—This procedure normally yields high specific activity products and requires halogenated or unsaturated precursors.

B. Reduction with Sodium Borohydride [³H]—This procedure is rather inexpensive and requires precursors containing reducible functional groups such as aldehydes, ketones, lactones, esters, and the like.

C. Reduction with Lithium Aluminum Hydride [³H]—This procedure offers products at almost theoretical specific activities. It also requires precursors containing reducible functional groups such as aldehydes, ketones, lactones, esters, and the like.

D. Tritium Gas Exposure Labeling—This procedure involves exposing precursors containing exchangeable protons to tritium gas in the presence of a suitable catalyst.

E. N-Methylation using Methyl Iodide [³H]—This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:

A. Sandmeyer and like reactions—This procedure transforms an aryl or heteroaryl amine into a diazonium salt, such as a tetrafluoroborate salt, and subsequently to ¹²⁵I labeled compound using Na¹²⁵I. A represented procedure was reported by Zhu, D.-G. and co-workers in J. Org. Chem. 2002, 67, 943-948.

B. Ortho ¹²⁵Iodination of phenols—This procedure allows for the incorporation of ¹²⁵I at the ortho position of a phenol as reported by Collier, T. L. and co-workers in J. Labeled Compd Radiopharm. 1999, 42, S264-S266.

C. Aryl and heteroaryl bromide exchange with ¹²⁵I. This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A represented procedure was reported by Bas, M.-D. and co-workers in J. Labeled Compd Radiopharm. 2001, 44, S280-S282.

A ligand of an olfactory GPCR may alternatively be identified by contacting a candidate agent with the olfactory GPCR in the presence of a labeled known ligand of the olfactory GPCR, wherein a decrease of binding of the labeled known ligand in the presence of the candidate agent is indicative of the candidate agent being a ligand of the olfactory GPCR.

Methods for Identifying Odorant Mimetics

The invention also provides methods for identifying odorant mimetics, where a mimetic is a synthetic or natural chemical compound that has similar, substantially the same or identical functional characteristics as a particular odorant, but has a different chemical structure to the odorant. In other words, the invention provides methods of identifying an odorant mimetic that “smells” the same as an odorant of interest, but does not have the same chemical structure as the odorant of interest. In general, these methods involve producing a library of olfactory GPCRs using the methods set forth above, identifying a set of olfactory GPCRs that are activated by an odorant of interest, and contacting the library of olfactory GPCRs with candidate agents to identify an agent that activates the same set of olfactory GPCRs. In most embodiments, an agent that activates the same set of olfactory GPCRs as an odorant of interest is a mimetic of the odorant of interest, i.e., should have a similar odor to the odorant of interest.

Accordingly, these methods usually involve producing a library (e.g., 100 or more, 200 or more, 300 or more, 400 or more, 500 or more, 600 or more, usually up to about 1000 or more) different olfactory GPCRs using the methods described above, and assessing the GPCRs to determine whether they are activated by an odorant of interest, e.g., a compound of known or unknown chemical structure that has a desirable smell or taste. In many embodiments, the odorant of interest will activate a set of olfactory GPCRs, where a set usually contains 2-50, 2-20 or 3-10 members. The set of olfactory GPCRs activated by a single odorant provides a “GPCR fingerprint”, where a single odorant is defined by the set of olfactory GPCRs that it activates. A mimetic for an odorant of interest may be identified by screening a library of candidate agents to identify an agent that has an identical or near identical GPCR fingerprint to that of the odorant of interest.

For example, an odorant mimetic can be identified so that is has a “fingerprint” of activated GPCRs similar to that of the odorant of interest, e.g., the mimetic activates about 60%, about 75%, about 80%, about 90%, about 95% of the GPCRs or GPCR activity as that activated by the odorant.

Accordingly, mimetics of an odorant of interest may be identified.

Biosensing Methods

The invention also provides a biosensor, where the biosensor is typically a plurality of macroglial cells producing a plurality of different olfactory GPCRs. In many embodiments, the cells are arrayed in an addressable format, wherein each address of the array contains macroglial cells producing a single recombinant olfactory GPCR. Typically, said plurality may be 2 or more, 5 or more, about 10 or more, about 20 or more, about 50 or more, about 100 or more, about 200 or more, about 300 or more, about 500 or more, about 1000 or more, or even up to about 10,000 or more. The biosensor may therefore contain about 5, about 10, about 20, about 30 or more, about 50 or more, about 100 or more, about 200 or more, usually up to 500 or more, usually up to about 1000 or more recombinant olfactory GPCRs. The olfactory GPCRs may be of known identity, or unknown identity, or a mixture thereof. The olfactory GPCRs may be derived from a single species or alternatively derived from 2, up to about 5, up to about 10, up to about 50, up to about 100, or up to about 1000 species of animal. In certain embodiments, the olfactory GPCRs are human.

The methods described herein involve binding of said macroglial cells producing a single recombinant olfactory GPCR to an “affinity substrate”. In certain embodiments, said affinity substrate is addressable. In particular embodiments, said addressable affinity substrate is spatially addressable. An affinity substrate is contains a solid, semi-solid, or insoluble support and is made from any material appropriate for binding of said recombinant macroglial cells and does not interfere with the detection method used. As will be appreciated by those in the art, the number of possible affinity substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, ceramics, and a variety of other polymers. In a preferred embodiment, the substrates allow optical detection and do not themselves appreciably fluoresce or emit light. In addition, as is known the art, the substrate may be coated with any number of materials, including polymers, such as dextrans, acrylamides, gelatins, agarose, biocompatible substances such as proteins including bovine and other mammalian serum albumin.

A “spatially addressable” affinity substrate has multiple, discrete, regions (e.g., multiple polypeptide of interest-binding regions) such that each region is at a particular predetermined location (an “address”). Multi-well microtiter plates are addressable (each well having an address), an array of capillary columns is addressable, an array of samples deposited onto a solid support (e.g., a nylon or nitrocellulose membrane) is addressable. Affinity substrates for use in the methods described herein typically have at least 4 or more, at least about 12, at least about 24, at least about 48, at least about 96 or at least about 384 or addressable regions. In particular embodiments, an affinity substrate is in an addressable format suitable for high throughput assays, e.g., a 24-, 48- 96- or 384-well format.

Such multi-well formats are suitable for use by robots, (e.g., pipetting robots), and other instrumentation (96- or 384-well format luminometers or fluorescence readers for determining reporter activity). By way of illustration and not limitation, reporter activity may be measured using a CCD camera-based illuminator.

In use, such biosensors are usually contacted with a sample, and activation of each of the recombinant olfactory GPCRs is assessed. The presence of an odorant of interest is detected by activation of a pre-determined subset of olfactory GPCRs, where the pre-determined subset of olfactory GPCRs corresponds to a previously determined “GPCR fingerprint” of that odorant. Accordingly, if a pre-determined subset of GPCRs for an odorant of interest is activated by the sample, then an odorant of interest is present in the sample.

In alternative use, such biosensors are usually contacted with an odorant, and activation of each of the recombinant GPCRs is assessed. Identification of a “fingerprint” for that odorant is assigned based on the subset of olfactory GPCRs activated. In a variation of said alternative use, said contacting may be carried out in the presence or more or more agonists to the olfactory GPCRs of the biosensor, with the subset of GPCRs activated by the odorant in the presence of said one more agonists representing another means of assigning a “fingerprint” to an odorant. It is envisioned that in the presence of an agonist, inverse agonist or antagonist activity of one or more olfactory GPCRs may be incorporated into a “fingerprint” of an odorant.

In certain embodiments, GPCR activation may be detected using a light-emitting reporter of GPCR activation. For example, any light-emitting reporter (e.g., a fluorescent reporter, etc.) assay may be used such as the luciferase/GFP based assays described below, or variations thereof, may be used for these assays.

In certain embodiments, the activation of one or more of the olfactory GPCRs to an odorant may be scored as being at a particular level, such as by exemplification and not limitation 0-10%, 11-25%, 26-50%, 51-75%, or 76-100% of a pre-determined maximum response. It is envisioned that a “fingerprint” of an odorant may be determined at least in part by the level of activation of one or more of the olfactory GPCRs.

Accordingly, the invention provides a light-emitting biosensor that contains an addressable array of macrogial cells containing olfactory GPCRs, where an odorant of interest may be detected by emission of a particular pattern of light from the biosensor.

In certain embodiments, the sample to be tested is an environmental test sample, e.g., a sample of a gas (such as a sample of a breathable atmosphere or a gas of unknown origin or composition), liquid (such as a sample of water or a liquid of unknown origin or composition), or any solid.

The biosensor methods described above find particular use in, for example: crime-scenes, where knowledge of a smell, for example, may lead to capture of a suspect for a crime; war zones (e.g., battlefields), where certain chemicals, e.g., biological/chemical warfare agents, may be detected; foodstuffs, where, e.g., certain contaminants or desirable or undesirable smells can be detected; and in the rational assignment of a particular olfactory GPCR or a particular subset of olfactory GPCR to either a desirable or undesirably olfactory sensation; and in laboratories where it is desirable to monitor noxious chemicals; and, in general, in any situation in which it is desirable to monitor or detect an odorant of interest.

Odorants of interest generally include any compound that can be detected by the olfactory GPCRs of the human olfactory system, e.g., any compound that can be detected by smelling. The odorant may be a purified compound or may be unpurified (e.g., of complex composition). Such odorants include, but are not limited to, aliphatic acids, alcohols, ketones, and esters; chemicals with aromatic, alicyclic, polycyclic and heterocyclic ring structures; and innumerable substituted chemicals of each of these types, as well as combinations thereof.

Utility

The subject methods of producing an olfactory GPCR find use in a variety of research and commercial applications, particularly those relating to food and fragrance.

In many applications, an item, e.g., a food or fragrance, may be improved, i.e., made more or less desirable, as needed, by addition of an olfactory GPCR modulatory agent identified using the methods described above. In general, such a modulator is usually mixed with the item, e.g., a foodstuff or fragrance such as a perfume, to improve the taste or smell of the item. In many embodiments, the modulator may be an inhibitor of olfactory GPCR activity, and therefore “mask” an unpleasant taste or smell. In other embodiments, the modulator may be an activator of certain olfactory GPCRs, and may be used to improve or add a new flavor or fragrance to the matter to which it is added. In other embodiments, the modulator may be an activator of certain olfactory GPCRs, and may be used to improve the efficacy of pesticides. In certain embodiments, it is advantageous to make the odor of some items such as poisons and medicines less desirable so that they are not accidentally consumed. In this case, an agent that provides an unpleasant odor may be discovered by the methods described above and added to those items.

In other applications, the cost of providing a desirable odorant (e.g., an odorant obtained from certain rare flowers and used as a starting material for many of today's perfumes) may be reduced by identifying mimetics of that odorant using the methods described above. In such embodiments, these mimetics may be manufactured at a price that is substantially less than that of the desirable odorant, and may be used to supplement, or replace the desirable odorant in an item e.g., a perfume, etc.

In other applications, detection of a particular odorant produced by an individual may have diagnostic or prognostic value as relates to a disease or disorder, wherein the elevation or reduction of the particular odorant has been associated with said disease or disorder.

Of course, a variety of individuals may be administered the olfactory GPCR modulators obtained using the methods described above. Generally such individuals are mammals or mammalian, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys), as well as mammals of commercial interest such as cows, sheep, pigs and horses. It is envisioned that non-mammalian animals may also be administered the olfactory GPCR modulators obtained using the methods described above. Exemplary and non-limiting non-mammalian animals include birds (e.g, chicken), reptiles, fish, arthropods, and insects (e.g., mosquito, ant, aphid, beetle, fly, wasp, bee, spider, or any insect which transmits a disease to human or non-human animals or which causes damage to crops or ornamental plants). In many embodiments, the individuals will be humans.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the subject invention.

### Example 1

### Expression of Olfactory GPCRs in Schwann Cells

Primary Rat Schwann Cell Isolation:

The preparation of Schwann cells was done as described previously (e.g., Hung, Int. J. Oncol. 20:475-82, 2002; Hung, Int. J. Oncol. 1999 14:409-15; Wood, Brain Res. 115:361-75, 1976; Wood, Ann. N.Y. Acad. Sci. 605:1-14, 1990; and Brockes, J. Exp. Biol. December; 95:215-30, 1981, etc). Briefly, sciatic nerves from P1 rat neonates were harvested and cells were maintained in Dulbecco's modified Eagle media supplemented with 10% heat-inactivated fetal bovine serum. Schwann cells were expanded with 2 uM forskolin and bovine pituitary extract (Sigma). The cells were grown until the third passage and frozen for storage.

Transient Transfection of Olfactory GPCRs:

Schwann cells at passage 5 were plated on poly-D-lysine coated 8-well chamber slides (Falcon) at 8×10⁴ cells per well. The Schwann cells were transfected with 0.5 ug of olfactory GPCR expression plasmid with Fugene6 reagent (Roche) and Optimem serum-free medium (Invitrogen). The transfected cells were kept at 37° in 5% CO₂ humidified incubator for four hours. Cells were washed with PBS and replaced with fresh growth media. After 24 hours, the cells were assayed for expression.

Expression Analysis of Olfactory GPCRs Determined by HA Staining:

The transfected cells were washed with PBSCM (PBS+0.5 mM Ca²⁺+1 mM MgCl₂) and fixed with 4% Formalin. Cells were quenched with 50 mM NH₄Cl/PBSCM and washed twice. Primary antibody anti-mouse HA (Roche) was diluted 1:1000 in blocking buffer (2% BSA in PBSCM w/o triton) and left on cells for 1 hour. After three washes with PBSCM, secondary antibody (Alexa 488-conjugated donkey anti-mouse IgG) 1:2000 and DAPI 1:2000 were left on cells for thirty minutes in the dark. Cells were washed 3 times with PBSCM and coversliped with fluorosave (Calbiochem) Cells analyzed by appropriate UV filters.

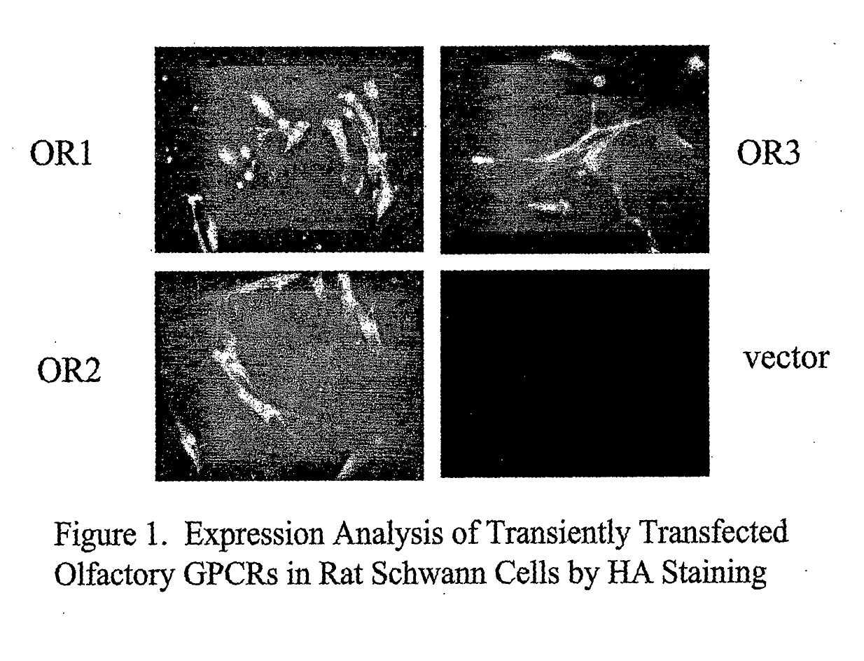

Cells producing olfactory GPCR on their surface were observed (see FIG. 1).

### Example 2

### GPCR Activation Assays

Receptor Expression: Transient transfection of macroglial cells may be carried out as described in Example 1 for primary rat Schwann cells. Stable transfection of a macroglial cell line may be carried out as described here.

Approximately 12×10⁶ macroglial cells are plated on a 15 cm tissue culture plate and grown in DME High Glucose Medium containing ten percent fetal bovine serum and one percent sodium pyruvate, L-glutamine, and antibiotics. Twenty-four hours following plating of the macroglial cells (or to 80% confluency), the cells are transfected using 12 μg of DNA. The 12 μg of DNA is combined with 60 μl of lipofectamine and 2 mL of DME High Glucose Medium without serum. The medium is aspirated from the plates and the cells are washed once with medium without serum. The DNA, lipofectamine, and medium mixture are added to the plate along with 10 ml of medium without serum. Following incubation at 37 degrees Celsius for four to five hours, the medium is aspirated and 25 ml of medium containing serum is added. Twenty-four hours following transfection, the medium is aspirated again, and fresh medium with serum is added. Forty-eight hours following transfection, the medium is aspirated and medium with serum is added containing geneticin (G418 drug) at a final concentration of 500 μg/ml. The transfected cells now undergo selection for positively transfected cells containing the G418 resistant gene. The medium is replaced every four to five days as selection occurs. During selection, cells are grown to create stable pools, or split for stable clonal selection.

Membrane Binding Assays: [³⁵S] GTPγS Assay: When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Activated receptors continue to exchange GDP for GTP. The non-hydrolyzable GTP analog, [³⁵S]GTPγS, can be utilized to demonstrate enhanced binding of [³⁵S]GTPγS to membranes expressing activated receptors. The advantage of using [³⁵S]GTPγS binding to measure activation is that: (a) it is generically applicable to all G protein-coupled receptors; (b) it is proximal at the membrane surface making it less likely to pick-up molecules which affect the intracellular cascade.

The assay utilizes the ability of G protein coupled receptors to stimulate [³⁵S]GTPγS binding to membranes expressing the relevant receptors. The assay can, therefore, be used in the direct identification method to screen candidate compounds to endogenous GPCRs and non-endogenous, constitutively activated GPCRs. The assay is generic and has application to drug discovery at all G protein-coupled receptors.

The [³⁵S]GTPγS assay is incubated in 20 mM HEPES and between 1 and about 20 mM MgCl₂ (this amount can be adjusted for optimization of results, although 20 mM is preferred) pH 7.4, binding buffer with between about 0.3 and about 1.2 nM [³⁵S]GTPγS (this amount can be adjusted for optimization of results, although 1.2 is preferred) and 12.5 to 75 μg membrane protein (this amount can be adjusted for optimization) and 10 μM GDP (this amount can be changed for optimization) for 1 hour. Wheatgerm agglutinin beads (25 μl; Amersham) are then added and the mixture incubated for another 30 minutes at room temperature. The tubes are then centrifuged at 1500×g for 5 minutes at room temperature and then counted in a scintillation counter.

Adenylyl Cyclase A Flash Plate™ Adenylyl Cyclase kit (New England Nuclear; Cat. No. SMP004A) designed for cell-based assays can be modified for use with crude plasma membranes. The Flash Plate wells can contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells were harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS are added to each plate. Cells were pipetted off the plate and the cell suspension was collected into a 50 ml conical centrifuge tube. Cells were then centrifuged at room temperature at 1,100 rpm for 5 min. The cell pellet was carefully re-suspended into an appropriate volume of PBS (about 3 ml/plate). The cells were then counted using a hemocytometer and additional PBS was added to give the appropriate number of cells (with a final volume of about 50 μl/well).

cAMP standards and Detection Buffer (comprising 1 μCi of tracer [¹²⁵I AMP (50 μl] to 11 ml Detection Buffer) was prepared and maintained in accordance with the manufacturer's instructions. Assay Buffer was prepared fresh for screening and contained 50 μl of Stimulation Buffer, 3 ul of test compound (12 μM final assay concentration) and 50 μl cells, Assay Buffer was stored on ice until utilized. The assay was initiated by addition of 50 μl of cAMP standards to appropriate wells followed by addition of 50 ul of PBSA to wells H-11 and H12. 50 μl of Stimulation Buffer was added to all wells. DMSO (or selected candidate compounds) was added to appropriate wells using a pin tool capable of dispensing 3 μl of compound solution, with a final assay concentration of 12 μM test compound and 100 μl total assay volume. The cells were then added to the wells and incubated for 60 min at room temperature. 100 μl of Detection Mix containing tracer cAMP was then added to the wells. Plates were then incubated additional 2 hours followed by counting in a Wallac MicroBeta scintillation counter. Values of cAMP/well were then extrapolated from a standard cAMP curve which was contained within each assay plate.

Cell-Based cAMP for Gi Coupled Target GPCRs: TSHR is a Gs coupled GPCR that causes the accumulation of cAMP upon activation. TSHR will be constitutively activated by mutating amino acid residue 623 (i.e., changing an alanine residue to an isoleucine residue). A Gi coupled receptor is expected to inhibit adenylyl cyclase, and, therefore, decrease the level of cAMP production, which can make assessment of cAMP levels challenging. An effective technique for measuring the decrease in production of cAMP as an indication of constitutive activation of a Gi coupled receptor can be accomplished by co-transfecting, most preferably, non-endogenous, constitutively activated TSHR (TSHR-A623I) (or an endogenous, constitutively active Gs coupled receptor) as a “signal enhancer” with a Gi linked target GPCR to establish a baseline level of cAMP. Upon creating a non-endogenous version of the Gi coupled receptor, this non-endogenous version of the target GPCR is then co-transfected with the signal enhancer, and it is this material that can be used for screening. We will utilize such approach to effectively generate a signal when a cAMP assay is used; this approach is preferably used in the direct identification of candidate compounds against Gi coupled receptors. It is noted that for a Gi coupled GPCR, when this approach is used, an inverse agonist of the target GPCR will increase the cAMP signal and an agonist will decrease the cAMP signal.

On day one, 2×10⁴ macroglial cells/well will be plated out. On day two, two reaction tubes will be prepared (the proportions to follow for each tube are per plate): tube A will be prepared by mixing 2 μg DNA of each receptor transfected into the mammalian cells, for a total of 4 μg DNA (e.g., pCMV vector, pCMV vector with mutated THSR (TSHR-A623I); TSHR-A623I and GPCR, etc.) in 1.2 ml serum free DMEM (Irvine Scientific, Irvine, Calif.); tube B will be prepared by mixing 120 μl lipofectamine (Gibco BRL) in 1.2 ml serum free DMEM. Tubes A and B will then be admixed by inversions (several times), followed by incubation at room temperature for 30-45 min. The admixture is referred to as the “transfection mixture”. Plated macroglial cells will be washed with 1×PBS, followed by addition of 10 ml serum free DMEM. 2.4 ml of the transfection mixture will then be added to the cells, followed by incubation for 4 hrs at 37° C./5% CO2. The transfection mixture will then be removed by aspiration, followed by the addition of 25 ml of DMEM/10% Fetal Bovine Serum. Cells will then be incubated at 37° C./5% CO2. After 24 hr incubation, cells will then be harvested and utilized for analysis.

A Flash Plate™ Adenylyl Cyclase kit (New England Nuclear; Cat. No. SMP004A) is designed for cell-based assays, however, can be modified for use with crude plasma membranes depending on the need of the skilled artisan. The Flash Plate wells will contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells will be harvested approximately twenty four hours after transient transfection. Media will be carefully aspirated off and discarded. 10 ml of PBS will be gently added to each dish of cells followed by careful aspiration. 1 ml of Sigma cell dissociation buffer and 3 ml of PBS will be added to each plate. Cells will be pipetted off the plate and the cell suspension will be collected into a 50 ml conical centrifuge tube. Cells will then be centrifuged at room temperature at 1,100 rpm for 5 min. The cell pellet will be carefully re-suspended into an appropriate volume of PBS (about 3 ml/plate). The cells will then be counted using a hemocytometer and additional PBS is added to give the appropriate number of cells (with a final volume of about 50 μl/well).

cAMP standards and Detection Buffer (comprising 1 μCi of tracer [¹²⁵I cAMP (50 μl] to 11 ml Detection Buffer) will be prepared and maintained in accordance with the manufacturer's instructions. Assay Buffer should be prepared fresh for screening and contained 50 μl of Stimulation Buffer, 3 μl of test compound (12 μM final assay concentration) and 50 μl cells, Assay Buffer can be stored on ice until utilized. The assay can be initiated by addition of 50 μl of cAMP standards to appropriate wells followed by addition of 50 μl of PBSA to wells H-11 and H12. Fifty μl of Stimulation Buffer will be added to all wells. Selected compounds (e.g., TSH) will be added to appropriate wells using a pin tool capable of dispensing 3 μl of compound solution, with a final assay concentration of 12 μM test compound and 100 μl total assay volume. The cells will then be added to the wells and incubated for 60 min at room temperature. 100 μl of Detection Mix containing tracer cAMP will then be added to the wells. Plates were then incubated additional 2 hours followed by counting in a Wallac MicroBeta scintillation counter. Values of cAMP/well will then be extrapolated from a standard cAMP curve which is contained within each assay plate.

Reporter-Based Assays: Cre-Luc Reporter Assay (Gs-associated receptors): macroglial cells are plated-out on 96 well plates at a density of 2×10⁴ cells per well and were transfected using Lipofectamine Reagent (BRL) the following day according to manufacturer instructions. A DNA/lipid mixture is prepared for each 6-well transfection as follows: 260 ng of plasmid DNA in 100 μl of DMEM were gently mixed with 2 μl of lipid in 100 μl of DMEM (the 260 ng of plasmid DNA consisted of 200 ng of a 8×CRE-Luc reporter plasmid, 50 ng of pCMV comprising endogenous receptor or non-endogenous receptor or pCMV alone, and 10 ng of a GPRS expression plasmid (GPRS in pcDNA3 (Invitrogen)). The 8×CRE-Luc reporter plasmid was prepared as follows: vector SRIF-β-gal was obtained by cloning the rat somatostatin promoter (−71/+51) at BglV-HindIII site in the pβgal-Basic Vector (Clontech). Eight (8) copies of cAMP response element were obtained by PCR from an adenovirus template AdpCF126CCRE8 (see, 7 Human Gene Therapy 1883 (1996)) and cloned into the SRIF-β-gal vector at the Kpn-BglV site, resulting in the 8×CRE-β-gal reporter vector. The 8×CRE-Luc reporter plasmid was generated by replacing the beta-galactosidase gene in the 8×CRE-β-gal reporter vector with the luciferase gene obtained from the pGL3-basic vector (Promega) at the HindIII-BamHI site. Following 30 min. incubation at room temperature, the DNA/lipid mixture was diluted with 400 μl of DMEM and 100 μl of the diluted mixture was added to each well. 100 μl of DMEM with 10% FCS were added to each well after a 4 hr incubation in a cell culture incubator. The following day the transfected cells were changed with 200 μl/well of DMEM with 10% FCS. Eight (8) hours later, the wells were changed to 100 μl/well of DMEM without phenol red, after one wash with PBS. Luciferase activity were measured the next day using the LucLite™ reporter gene assay kit (Packard) following manufacturer instructions and read on a 1450 MicroBeta™ scintillation and luminescence counter (Wallac).

AP1 reporter assay (Gq-associated receptors) A method to detect Gq stimulation depends on the known property of Gq-dependent phospholipase C to cause the activation of genes containing API elements in their promoter. A Pathdetect™ AP-1 cis-Reporting System (Stratagene, Catalogue # 219073) can be utilized following the protocol set forth above with respect to the CREB reporter assay, except that the components of the calcium phosphate precipitate were 410 ngpAP1-Luc, 80 ng pCMV-receptor expression plasmid, and 20 ng CMV-SEAP.

SRF-Luc Reporter Assay (Gq-associated receptors): One method to detect G4 stimulation depends on the known property of Gq-dependent phospholipase C to cause the activation of genes containing serum response factors in their promoter. A Pathdetect™ SRF-Luc-Reporting System (Stratagene) can be utilized to assay for Gq coupled activity in, e.g., COS7 cells. Cells are transfected with the plasmid components of the system and the indicated expression plasmid encoding endogenous or non-endogenous GPCR using a Mammalian Transfection™ Kit (Stratagene, Catalogue #200285) according to the manufacturer's instructions. Briefly, 410 ng SRF-Luc, 80 ng pCMV-receptor expression plasmid and 20 ng CMV-SEAP (secreted alkaline phosphatase expression plasmid; alkaline phosphatase activity is measured in the media of transfected cells to control for variations in transfection efficiency between samples) are combined in a calcium phosphate precipitate as per the manufacturer's instructions. Half of the precipitate is equally distributed over 3 wells in a 96-well plate, kept on the cells in a serum free media for 24 hours. The last 5 hours the cells are incubated with a selected compound. Cells are then lysed and assayed for luciferase activity using a Luclite™ Kit (Packard, Cat. # 6016911) and “Trilux 1450 Microbeta” liquid scintillation and luminescence counter (Wallac) as per the manufacturer's instructions. The data can be analyzed using GraphPad Prism™ 2.0a (GraphPad Software Inc.)

Intracellular inositol 1,4,5-triphosphate (IP3) Accumulation Assay (G_{q}-associated receptors): On day 1, cells comprising the receptors (endogenous and/or non-endogenous) can be plated onto 24 well plates, usually 1×10⁵ cells/well (although his number can be optimized. On day 2 cells can be transfected by firstly mixing 0.25 μg DNA in 50 μl serum free DMEM/well and 2 μl lipofectamine in 50 μl serumfree DMEM/well. The solutions are gently mixed and incubated for 15-30 min at room temperature. Cells are washed with 0.5 ml PBS and 400 μl of serum free media is mixed with the transfection media and added to the cells. The cells are then incubated for 34 hrs at 37° C./5% CO₂ and then the transfection media is removed and replaced with 1 ml/well of regular growth media.

On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of ³H-myo-inositol/well and the cells are incubated for 16-18 hrs o/n at 37° C./5% CO₂. On Day 4 the cells are washed with 0.5 ml PBS and 0.45 ml of assay medium is added containing inositol-free/serum free media 10 μM pargyline 10 mM lithium chloride or 0.4 ml of assay medium and 50 μl of 10× ketanserin (ket) to final concentration of 10 μM. The cells are then incubated for 30 min at 37° C. The cells are then washed with 0.5 ml PBS and 200 μl of fresh/ice cold stop solution (1M KOH; 18 mM Na-borate; 3.8 mM EDTA) is added/well. The solution is kept on ice for 5-10 min or until cells were lysed and then neutralized by 200 μl of fresh/ice cold neutralization sol. (7.5% HCL).

The lysate is then transferred into 1.5 ml eppendorf tubes and 1 ml of chloroform/methanol (1:2) is added/tube. The solution is vortexed for 15 sec and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3M ammonium formate and rinsed twice with dd H₂O and stored at 4° C. in water.

Fluorometric Imaging Plate Reader (FLIPR) Assay for the Measurement of Intracellular Calcium Concentration: Target Receptor (experimental) and pCMV (negative control) stably transfected cells from respective clonal lines are seeded into poly-D-lysine pretreated 96-well plates (Becton-Dickinson, #356640) at 5.5×10⁴ cells/well with complete culture medium (DMEM with 10% FBS, 2 mM L-glutamine, 1 mM sodium pyruvate) for assay the next day. To prepare Fluo4-AM (Molecular Probe, #F14202) incubation buffer stock, 1 mg Fluo4-AM is dissolved in 467 μl DMSO and 467 μl Pluoronic acid (Molecular Probe, #P3000) to give a 1 mM stock solution that can be stored at −20° C. for a month. Fluo4-AM is a fluorescent calcium indicator dye.

Candidate compounds are prepared in wash buffer (1×HBSS/2.5 mM Probenicid/20 mM HEPES at pH 7.4).

At the time of assay, culture medium is removed from the wells and the cells are loaded with 100 μl of 4 μM Fluo4-AM/2.5 mM Probenicid (Sigma, #P8761)/20 mM HEPES/complete medium at pH 7.4. Incubation at 37° C./5% CO₂ is allowed to proceed for 60 min.

After the 1 hr incubation, the Fluo4-AM incubation buffer is removed and the cells are washed 2× with 100 μl wash buffer. In each well is left 100 μl wash buffer. The plate is returned to the incubator at 37° C./5% CO₂ for 60 min.

FLIPR (Fluorometric Imaging Plate Reader; Molecular Device) is programmed to add 50 μl candidate compound on the 30^{th} second and to record transient changes in intracellular calcium concentration ([Ca²⁺]) evoked by the candidate compound for another 150 seconds. Total fluorescence change counts are used to determine agonist activity using the FLIPR software. The instrument software normalizes the fluorescent reading to give equivalent initial readings at zero.

In some embodiments, the cells comprising Target Receptor further comprise promiscuous G alpha 15/16 or the chimeric Gq/Gi alpha unit.

Although the foregoing provides a FLIPR assay for agonist activity using stably transfected cells, a person of ordinary skill in the art would readily be able to modify the assay in order to characterize antagonist activity. Said person of ordinary skill in the art would also readily appreciate that, alternatively, transiently transfected cells could be used.

It is evident from the above results and discussion that the subject invention provides an important new means for producing olfactory GPCRs. In particular, the subject invention provides a system for screening chemical agent libraries to find olfactory GPCR modulators. As such, the subject methods and systems find use in a variety of different applications, including research, food and fragrance improvement, and other applications. Accordingly, the present invention represents a significant contribution to the art.

TABLE 1

| | | | | | | | |
|---|---|---|---|---|---|---|---|
| OR51B2 | P47884 | Q8VGE1 | Q8VG94 | Q8VG21 | Q8VEZ0 | Q8VGS5 | Q8VGU6 |
| Q9Y5P0 | P58170 | Q8VGJ1 | Q8VG95 | Q8VG34 | Q8VEZ9 | Q8VGS6 | Q8VGU7 |
| Q9H255 | P30953 | Q8VGJ6 | Q8VGA0 | Q8VG41 | Q8VF01 | Q8VGS7 | Q8VGU8 |
| O88628 | P47887 | Q8VGP8 | Q8VGA9 | Q8VG47 | Q8VF12 | Q8VGS8 | Q8VGX1 |
| Q9H343 | O43749 | Q8VGT6 | Q8VGB1 | Q8VG57 | Q8VF13 | Q8VGS9 | Q8VGX5 |
| Q9H344 | P47890 | Q8VGT7 | Q8VGB2 | Q8VG58 | Q8VF14 | Q8VGT8 | Q9JHB2 |
| Q9H341 | O60431 | Q8VGT9 | Q8VGB6 | Q8VG59 | Q8VF15 | Q8VGU3 | Q9JHW3 |
| Q9UKL2 | Q15612 | Q920G5 | Q8VGB7 | Q8VG60 | Q8VF19 | Q8VGY2 | Q9JM16 |
| Q96RD2 | P23269 | Q9EPG3 | Q8VGC8 | Q8VG61 | Q8VF22 | Q8VH07 | O35184 |
| Q9H346 | P23274 | Q9EPG4 | Q8VGD6 | Q8VG62 | Q8VF25 | Q8VH08 | Q96RD0 |
| Q96RD3 | P23273 | Q9JKA6 | Q8VGD7 | Q8VG63 | Q8VF34 | Q8VH10 | Q96RC9 |
| Q8NGF0 | P23266 | Q9GZK7 | Q8VGD8 | Q8VG73 | Q8VF36 | Q920P1 | Q15620 |
| Q8NGF1 | P23271 | Q8NG94 | Q8VGD9 | Q8VG74 | Q8VF50 | Q920P2 | Q8WZ84 |
| Q8NGF3 | P23272 | Q8NGC1 | Q8VGI0 | Q8VG82 | Q8VF51 | Q923Q6 | Q9GZM6 |
| Q8NGH5 | P30955 | Q8NGC7 | Q8VGJ5 | Q8VG86 | Q8VF52 | Q923Q8 | Q63395 |
| Q8NGH6 | P70526 | Q8NGC9 | Q8VGL6 | Q8VGE4 | Q8VF53 | Q9EQ84 | Q8N0Y1 |
| Q8NGH7 | Q62942 | Q8NH07 | Q8VGL7 | Q8VGE5 | Q8VF54 | Q9EQ85 | Q8NG78 |
| Q8NGH8 | Q8NGA1 | Q8VEV3 | Q8VGP4 | Q8VGE6 | Q8VF59 | Q9EQ86 | Q8NG88 |
| Q8NGH9 | Q8NGQ3 | Q8VEV4 | Q8VGP5 | Q8VGE7 | Q8VF60 | Q9EQ87 | Q8NGG6 |
| Q8NGI0 | Q8NGR2 | Q8VF70 | Q8VGP6 | Q8VGE8 | Q8VF61 | Q9EQ88 | Q8NGG7 |
| Q8NGI1 | Q8NGR5 | Q8VFC3 | Q8VGT5 | Q8VGE9 | Q8VF65 | Q9QY00 | Q8NGG8 |
| Q8NGI2 | Q8NGR7 | Q8VFD8 | Q8VGU0 | Q8VGF1 | Q8VF66 | Q9WU91 | Q8NGG9 |
| Q8NGI3 | Q8NGR8 | Q8VFE3 | Q8VGW6 | Q8VGF2 | Q8VF67 | O13036 | Q8NGH0 |
| Q8NGJ2 | Q8NGR9 | Q8VFT6 | Q8VGW9 | Q8VGF3 | Q8VF68 | O57597 | Q8NGH1 |
| Q8NGJ3 | Q8NGS0 | Q8VFT7 | Q8VGX0 | Q8VGF4 | Q8VF71 | O95222 | Q8NGH2 |
| Q8NGJ4 | Q8NGS1 | Q8VFT8 | Q8VGX2 | Q8VGF5 | Q8VF72 | O95007 | Q8NGM9 |
| Q8NGJ5 | Q8NGS2 | Q8VFT9 | Q920Z2 | Q8VGF6 | Q8VF73 | O70269 | Q8VEY0 |
| Q8NGJ6 | Q8NGS3 | Q9WU86 | Q9D3U9 | Q8VGF7 | Q8VF74 | O70270 | Q8VF23 |
| Q8NGJ7 | Q8NGZ1 | P58182 | Q9D4F9 | Q8VGF8 | Q8VF75 | O70271 | Q8VF62 |
| Q8NGJ8 | Q8NH92 | Q9UGF7 | Q9EP55 | Q8VGF9 | Q8VF76 | P23267 | Q8VF63 |
| Q8NGJ9 | Q8NH93 | Q8NHA7 | Q9EP67 | Q8VGG0 | Q8VF77 | P23270 | Q8VF64 |
| Q8NGK0 | Q8NH94 | Q8VG96 | Q9EPF5 | Q8VGG1 | Q8VFC4 | Q8C0U2 | Q8VF78 |
| Q8NGK1 | Q8NHA8 | Q920Y8 | Q9EPF6 | Q8VGG2 | Q8VFC5 | Q8K4Z9 | Q8VFB3 |
| Q8NGK2 | Q8VET9 | Q920Y9 | Q9EPF7 | Q8VGG7 | Q8VFC9 | Q8K501 | Q8VFB4 |
| Q8NGK3 | Q8VEU7 | Q920Z0 | Q9EPF8 | Q8VGG8 | Q8VFD0 | Q8NGC5 | Q8VFB5 |
| Q8NGK4 | Q8VBY8 | O95047 | Q9EPF9 | Q8VGH7 | Q8VFD1 | Q8NGD9 | Q8VFB6 |
| Q8NGK5 | Q8VEZ6 | Q9GZK3 | Q9EPG0 | Q8VGH8 | Q8VFD2 | Q8NGE1 | Q8VFD7 |
| Q8NGK6 | Q8VEZ7 | O76000 | Q9EPG5 | Q8VGH9 | Q8VFD3 | Q8NGE2 | Q8VFN2 |
| Q8NGM7 | Q8VF79 | P58173 | Q9EPG6 | Q8VGM3 | Q8VFG0 | Q8NGM8 | Q8VFN3 |
| Q8NH53 | Q8VFA1 | O95371 | Q9EPV1 | Q8VGM4 | Q8VFG1 | Q8NGN1 | Q8VFN4 |
| Q8NH55 | Q8VFD9 | Q9H210 | Q9GZK1 | Q8VGM5 | Q8VFG5 | Q8NGQ2 | Q8VFN5 |
| Q8NH56 | Q8VFE0 | Q13607 | Q9GZK6 | Q8VGM6 | Q8VFG6 | Q8NGT5 | Q8VG15 |
| Q8NH57 | Q8VFE1 | O95006 | Q9QW34 | Q8VGM7 | Q8VFJ7 | Q8NGU2 | Q8VG16 |
| Q8NH60 | Q8VFE4 | Q9H205 | Q9QW38 | Q8VGM8 | Q8VFJ8 | Q8NGW0 | Q8VG17 |
| Q8NH61 | Q8VFE5 | Q9GZK4 | Q9QZ17 | Q8VGM9 | Q8VFJ9 | Q8NGW1 | Q8VG50 |
| Q8NH63 | Q8VFE6 | O95918 | Q9QZ18 | Q8VGN0 | Q8VFK0 | Q8NGW6 | Q8VG51 |
| Q8NH64 | Q8VFM9 | Q15062 | Q9QZ19 | Q8VGN1 | Q8VFK1 | Q8NGX0 | Q8VG52 |
| Q8NH67 | Q8VFP4 | O76002 | Q9QZ20 | Q8VGN2 | Q8VFK2 | Q8NGX8 | Q8VG53 |
| Q8NH68 | Q8VFP5 | O76001 | Q9QZ21 | Q8VGN3 | Q8VFK3 | Q8NGX9 | Q8VG54 |
| Q8NH76 | Q8VFP6 | Q9NQN1 | Q9QZ22 | Q8VGN4 | Q8VFK4 | Q8NGY2 | Q8VG55 |
| Q8NH78 | Q8VFP7 | O43869 | Q9ROZ2 | Q8VGN5 | Q8VFK5 | Q8NGY3 | Q8VG56 |
| Q8TCB6 | Q8VFP8 | Q9Y3N9 | P47881 | Q8VGN6 | Q8VFK6 | Q8NGY4 | Q8VG67 |
| Q8VBV9 | Q8VFP9 | O35434 | P47893 | Q8VGN7 | Q8VFK7 | Q8NGY5 | Q8VG68 |
| Q8VEW7 | Q8VFT2 | O95499 | P47888 | Q8VGN8 | Q8VFK8 | Q8NGY6 | Q8VG69 |
| Q8VEW8 | Q8VFY1 | P23275 | P47883 | Q8VGN9 | Q8VFK9 | Q8NGZ6 | Q8VG70 |
| Q8VEX9 | Q8VGB9 | Q95156 | Q8VFX6 | Q8VGP0 | Q8VFL0 | Q8NH40 | Q8VG71 |
| Q8VF02 | Q8VGG9 | Q63394 | Q8VFX7 | Q8VGP1 | Q8VFL1 | Q8NH79 | Q8VG75 |
| Q8VF03 | Q8VGH0 | Q8N349 | Q8VFX8 | Q8VGP2 | Q8VFL2 | Q8VEU0 | Q8VG76 |
| Q8VF06 | Q8VGH1 | Q8N628 | Q8VFX9 | Q8VGP3 | Q8VFL4 | Q8VEU1 | Q8VG80 |
| Q8VF07 | Q8VGI1 | Q8NG76 | Q8VGR1 | Q9QW37 | Q8VFL5 | Q8VEW0 | Q8VG89 |
| Q8VF08 | Q8VGI2 | Q8NG77 | Q8VGR2 | Q9R0K1 | Q8VFL6 | Q8VEX2 | Q8VG90 |
| Q8VF09 | Q8VGI3 | Q8NG80 | Q9TSM7 | Q9R0K2 | Q8VFL7 | Q8VEX8 | Q8VG92 |
| Q8VF27 | Q8VGJ7 | Q8NG81 | Q9TSM8 | Q9R0K3 | Q8VFL8 | Q8VF24 | Q8VG93 |
| Q8VF28 | Q8VGJ8 | Q8NG82 | Q9TU88 | Q9R0K4 | Q8VFL9 | Q8VF26 | Q8VGB4 |
| Q8VFZ7 | Q8VGJ9 | Q8NG83 | Q9TU89 | Q9R0K5 | Q8VFM0 | Q8VF30 | Q8VGC9 |
| Q8VG01 | Q8VGK0 | Q8NG84 | Q9TU97 | Q96R09 | Q8VFM1 | Q8VF31 | Q8VGD0 |
| Q8VG18 | Q8VGK1 | Q8NG85 | Q9TUA0 | Q96R08 | Q8VFN7 | Q8VF33 | Q8VGD1 |
| Q8VG19 | Q8VGK2 | Q8NG86 | Q9TUA4 | O95221 | Q8VFN8 | Q8VF82 | Q8VGD2 |
| Q8VG22 | Q8VGK3 | Q8NG97 | Q15615 | Q13606 | Q8VFN9 | Q8VFB7 | Q8VGD3 |
| Q8VG23 | Q8VGK4 | Q8NGH3 | P58180 | Q8WZ92 | Q8VFP1 | Q8VFE7 | Q8VGD4 |
| Q8VG24 | Q8VGK5 | Q8NGH4 | O95013 | Q8WZ94 | Q8VFQ3 | Q8VFH3 | Q8VGD5 |
| Q8VG25 | Q8VGK6 | Q8NGS4 | Q8IXE1 | Q9UGF5 | Q8VFQ4 | Q8VFH4 | Q8VGE2 |
| Q8VG26 | Q8VGK7 | Q8NGS5 | Q8K4Z8 | Q9UGF6 | Q8VFQ5 | Q8VFH5 | Q8VGE3 |
| Q8VG28 | Q8VGK8 | Q8NGS6 | Q8K500 | O77756 | Q8VFQ6 | Q8VFH6 | Q8VH09 |
| Q8VG77 | Q8VGK9 | Q8NGS7 | Q8N0Y3 | O77757 | Q8VFQ7 | Q8VFH7 | Q9EQ89 |
| Q8VG78 | Q8VGL0 | Q8NGS8 | Q8NGA8 | O77758 | Q8VFQ8 | Q8VFH8 | Q9EQ90 |
| Q8VG79 | Q8VGP7 | Q8NGS9 | Q8NGB1 | Q95154 | Q8VFQ9 | Q8VFH9 | Q9EQ91 |
| Q8VG84 | Q8VGR3 | Q8NGT0 | Q8NGB2 | P37067 | Q8VFR0 | Q8VFI0 | Q9EQ92 |
| Q8VG85 | Q8VGR4 | Q8NGT1 | Q8NGB4 | Q95155 | Q8VFR1 | Q8VFI1 | Q9EQ93 |
| Q8VGA1 | Q8VGR5 | Q8NGT2 | Q8NGB6 | P37068 | Q8VFR2 | Q8VFI2 | Q9EQ94 |
| Q8VGU9 | Q8VGR6 | Q8NGT6 | Q8NGB8 | P37069 | Q8VFR3 | Q8VFI3 | Q9EQ95 |
| Q8VGV0 | Q8VGR7 | Q8NGT7 | Q8NGB9 | P37070 | Q8VFR4 | Q8VFI4 | Q9EQ96 |
| Q8VGV1 | Q8VGT0 | Q8NGT8 | Q8NGC2 | P37071 | Q8VFR5 | Q8VFI5 | Q9EQ97 |
| Q8VGV2 | Q8VGT1 | Q8NGT9 | Q8NGC6 | P37072 | Q8VFR6 | Q8VFN6 | Q9EQ98 |
| Q8VGV3 | Q8VGT2 | Q8NGU4 | Q8NGD0 | Q62943 | Q8VFR7 | Q8VFP0 | Q9EQ99 |
| Q8VGV4 | Q8VGT3 | Q8NGV0 | Q8NGD1 | Q62944 | Q8VFR8 | Q8VFP2 | Q9EQA0 |
| Q8VGV5 | Q920Y6 | Q8NGV1 | Q8NGD2 | Q8C0S2 | Q8VFR9 | Q8VFU0 | Q9EQA1 |
| Q8VGV6 | Q920Y7 | Q8NGV4 | Q8NGD3 | Q8IVL3 | Q8VFS0 | Q8VFU1 | Q9EQA2 |
| Q8VGV8 | Q9JHE2 | Q8NGV5 | Q8NGD4 | Q8IXE7 | Q8VFS7 | Q8VFU2 | Q9EQA3 |
| Q8VGV9 | Q9QW35 | Q8NGW7 | Q8NGD5 | Q8N0Y5 | Q8VFS8 | Q8VFU5 | Q9EQA4 |
| Q8VGW0 | Q9TQX4 | Q8NGX1 | Q8NGD6 | Q8N127 | Q8VFS9 | Q8VFY9 | Q9EQA5 |
| Q8VGW1 | Q9TSN0 | Q8NGX2 | Q8NGE8 | Q8N146 | Q8VFT0 | Q8VFZ0 | Q9EQA6 |
| Q8VGW2 | Q9TU84 | Q8NGY9 | Q8NGF8 | Q8N162 | Q8VFU3 | Q8VFZ1 | Q9EQA7 |
| Q8VGW3 | Q9TU86 | Q8NGZ0 | Q8NGF9 | Q8NG75 | Q8VFU4 | Q8VFZ2 | Q9EQA8 |
| Q8VGW4 | Q9TU90 | Q8NGZ4 | Q8NGI4 | Q8NGC0 | Q8VFU6 | Q8VFZ8 | Q9EQA9 |
| Q8VGW5 | Q9TU92 | Q8NGZ5 | Q8NGI6 | Q8NGC3 | Q8VFU7 | Q8VFZ9 | Q9EQB0 |
| Q8VGX3 | Q9TU93 | Q8NGZ9 | Q8NGJ1 | Q8NGC4 | Q8VFV2 | Q8VG27 | Q9EQB1 |
| Q8VGX4 | Q9TU94 | Q8NH00 | Q8NGL6 | Q8NGE7 | Q8VFV3 | Q8VG29 | Q9EQB2 |
| Q8VGX6 | Q9TU95 | Q8NH01 | Q8NGL7 | Q8NGE9 | Q8VFV4 | Q8VG33 | Q9EQB3 |
| Q8VGX7 | Q9TU99 | Q8NH02 | Q8NGL8 | Q8NGF4 | Q8VFV5 | Q8VG45 | Q9EQB4 |
| Q8VGX8 | Q9TUA1 | Q8NH04 | Q8NGL9 | Q8NGF5 | Q8VFV6 | Q8VG46 | Q9EQB5 |
| Q8VGX9 | Q9TUA2 | Q8NH16 | Q8NGM0 | Q8NGF7 | Q8VFV7 | Q8VG64 | Q9EQB6 |
| Q8VGY0 | Q9TUA3 | Q8NH95 | Q8NGN0 | Q8NGG0 | Q8VFV8 | Q8VGC2 | Q9EQB7 |
| Q8VGY1 | Q9TUA6 | Q8NHA4 | Q8NGN8 | Q8NGG2 | Q8VFV9 | Q8VGG4 | Q9EQB8 |
| Q8VGY3 | Q9TUA7 | Q8NHA6 | Q8NGN9 | Q8NGG3 | Q8VFW0 | Q8VGC5 | Q9EQG1 |
| Q8VGY4 | Q9TUA8 | Q8NHC8 | Q8NGP0 | Q8NGG4 | Q8VFW1 | Q8VGH3 | Q9ERU6 |
| Q8VGY5 | Q9TUA9 | Q8VES9 | Q8NH05 | Q8NGG5 | Q8VFW2 | Q8VGH4 | Q9QW36 |
| Q8VGY6 | Q9UDD9 | Q8VET2 | Q8NH21 | Q8NGI8 | Q8VFW3 | Q8VGH5 | Q8N148 |
| Q8VGY7 | O70265 | Q8VEV0 | Q8NH37 | Q8NGI9 | Q8VFW4 | Q8VGH6 | Q8NG79 |
| Q8VGY8 | O70266 | Q8VEV1 | Q8NH41 | Q8NGJ0 | Q8VFW5 | Q8VGI7 | Q8NG92 |
| Q8VGY9 | O70267 | Q8VEV9 | Q8NH42 | Q8NGK9 | Q8VFW6 | Q8VGI8 | Q8NGE0 |
| Q8VGZ0 | O70268 | Q8VEW4 | Q8NH43 | Q8NGL0 | Q8VFW7 | Q8VGI9 | Q8NGR1 |
| Q8VGZ1 | P58181 | Q8VEW9 | Q8NH49 | Q8NGL1 | Q8VFW8 | Q8VGJ0 | Q8NGR6 |
| Q8VGZ2 | Q9H209 | Q8VEY4 | Q8NH70 | Q8NGL2 | Q8VFW9 | Q8VGJ2 | Q8NGV6 |
| Q8VGZ3 | Q9H207 | Q8VEY6 | Q8NH72 | Q8NGL3 | Q8VFX0 | Q8VGJ3 | Q8NGV7 |
| Q8VGZ4 | Q96KK4 | Q8VEY7 | Q8NH73 | Q8NGL4 | Q8VFX1 | Q8VGL1 | Q8NGZ3 |
| Q8VGZ5 | Q9Y4A9 | Q8VF05 | Q8NH83 | Q8NGL5 | Q8VFX2 | Q8VGU1 | Q8NH08 |
| Q8VGZ6 | O60403 | Q8VF17 | Q8NH84 | Q8NGN2 | Q8VFX3 | Q8VGU4 | Q8NH09 |
| Q8VGZ7 | O60404 | Q8VF18 | Q8VET0 | Q8NGN3 | Q8VFX4 | Q8VGU5 | Q8NH14 |
| Q8VGZ8 | P30954 | Q8VF37 | Q8VET4 | Q8NGN4 | Q8VFX5 | Q8VGW8 | Q8NH44 |
| Q8VGZ9 | Q62007 | Q8VF44 | Q8VBX0 | Q8NGN5 | Q8VFZ3 | Q924H8 | Q8NHB7 |
| Q8VH00 | Q8CG22 | Q8VF69 | Q8VEX1 | Q8NGN6 | Q8VG00 | Q9EPG1 | Q8NHB8 |
| Q8VH01 | Q8NGA5 | Q8VF80 | Q8VEX3 | Q8NGN7 | Q8VG02 | Q9EPG2 | Q8NHC5 |
| Q8VH02 | Q8NGA6 | Q8VF81 | Q8VEX7 | Q8NGP2 | Q8VG03 | Q9EPV0 | Q8NHC6 |
| Q8VH03 | Q8NGE3 | Q8VF87 | Q8VEY5 | Q8NGP3 | Q8VG04 | Q9H206 | Q8VET6 |
| Q8VH04 | Q8NGE5 | Q8VF88 | Q8VEZ1 | Q8NGP4 | Q8VG05 | Q9QWU6 | Q8VET7 |
| Q8VH05 | Q8NGF6 | Q8VF89 | Q8VEZ2 | Q8NGP6 | Q8VG06 | Q9Z1V0 | Q8VEX5 |
| Q8VH06 | Q8NGI7 | Q8VF92 | Q8VEZ3 | Q8NGP8 | Q8VG07 | P34987 | Q8VEX6 |
| Q8VH11 | Q8NGM4 | Q8VFA2 | Q8VF10 | Q8NGP9 | Q8VG08 | Q15622 | Q8VF04 |
| Q8VH12 | Q8NGQ4 | Q8VFA3 | Q8VF11 | Q8NGQ0 | Q8VG09 | O76100 | Q8VF16 |
| Q8VH13 | Q8NGX3 | Q8VFA4 | Q8VF21 | Q8NGQ1 | Q8VG11 | O14581 | Q8VF32 |
| Q8VH14 | Q8NGX5 | Q8VFA5 | Q8VF29 | Q8NGQ5 | Q8VG13 | O76099 | Q8VF35 |
| Q8VH15 | Q8NGX6 | Q8VFA6 | Q8VF38 | Q8NGQ6 | Q8VG20 | O60412 | Q8VF42 |
| Q8VH16 | Q8NGY0 | Q8VFA7 | Q8VF39 | Q8NGR3 | Q8VG30 | P23268 | Q8VF43 |
| Q8VH17 | Q8NGY1 | Q8VFA8 | Q8VF40 | Q8NGR4 | Q8VG35 | P23265 | Q8VF93 |
| Q8VH18 | Q8NH19 | Q8VFA9 | Q8VF41 | Q8NGZ2 | Q8VG36 | Q95157 | Q8VFB8 |
| Q8VH19 | Q8NH36 | Q8VFB2 | Q8VF45 | Q8NH10 | Q8VG37 | Q8N133 | Q8VFB9 |
| Q8VH20 | Q8NH74 | Q8VFC1 | Q8VF46 | Q8NH18 | Q8VG38 | Q8NG95 | Q8VFC0 |
| Q8VH21 | Q8NHC4 | Q8VFC2 | Q8VF47 | Q8NH48 | Q8VG39 | Q8NG98 | Q8VFE8 |
| Q8VH22 | Q8VBE9 | Q8VFD4 | Q8VF48 | Q8NH50 | Q8VG40 | Q8NG99 | Q8VFE9 |
| Q924X8 | Q8VES6 | Q8VFD5 | Q8VF56 | Q8NH51 | Q8VG42 | Q8NGA0 | Q8VFP3 |
| Q99NH4 | Q8VES7 | Q8VFD6 | Q8VF57 | Q8NH69 | Q8VG43 | Q8NGA2 | Q8VFY0 |
| Q9EPN8 | Q8VEU3 | Q8VFF0 | Q8VF58 | Q8NH80 | Q8VG44 | Q8NH99 | Q8VFY6 |
| Q9EPN9 | Q8VEV2 | Q8VFG2 | Q8VF83 | Q8NH81 | Q8VG65 | Q8NHB5 | Q8VFY7 |
| Q9EQQ5 | Q8VEW1 | Q8VFG3 | Q8VF84 | Q8NH85 | Q8VG66 | Q8NHC1 | Q8VG48 |
| Q9EQQ6 | Q8VEX4 | Q8VFG4 | Q8VF85 | Q8NH86 | Q8VG81 | Q8VET8 | Q8VGH2 |
| Q9EQQ7 | Q8VEY1 | Q8VFG7 | Q8VF86 | Q8NH87 | Q8VG83 | Q8VEW3 | Q8VGJ4 |
| Q9GKV8 | Q8VEZ4 | Q8VFG8 | Q8VF90 | Q8NH88 | Q8VG91 | Q8VEY9 | Q8VGL2 |
| Q9H2C5 | Q8VEZ5 | Q8VFG9 | Q8VF91 | Q8NH89 | Q8VG97 | Q8VFF1 | Q8VGL3 |
| Q9H2C6 | Q8VEZ8 | Q8VFH0 | Q8VF94 | Q8NH90 | Q8VGA2 | Q8VFF2 | Q8VGL4 |
| Q9H2C8 | Q8VF00 | Q8VFH1 | Q8VF95 | Q8NH91 | Q8VGA3 | Q8VFF3 | Q8VGL5 |
| Q9H339 | Q8VF20 | Q8VFH2 | Q8VF96 | Q8NHC7 | Q8VGA4 | Q8VFF4 | Q8VGL8 |
| Q9H340 | Q8VF55 | Q8VFL3 | Q8VF97 | Q8VES8 | Q8VGA5 | Q8VFF5 | Q8VGL9 |
| Q9H342 | Q8VFE2 | Q8VFM2 | Q8VF98 | Q8VET1 | Q8VGA6 | Q8VFF6 | Q8VGM0 |
| Q9H345 | Q8VFM7 | Q8VFM3 | Q8VF99 | Q8VET3 | Q8VGA7 | Q8VFF7 | Q8VGM1 |
| Q9WU88 | Q8VFQ0 | Q8VFM4 | Q8VFA0 | Q8VET5 | Q8VGA8 | Q8VFI7 | Q8VGM2 |
| Q9WU89 | Q8VFQ2 | Q8VFM5 | Q8VFB0 | Q8VEU2 | Q8VGB0 | Q8VFI8 | Q8VGP9 |
| Q9WU90 | Q8VFS1 | Q8VFM6 | Q8VFB1 | Q8VEU4 | Q8VGB3 | Q8VFJ0 | Q8VGQ0 |
| Q9WU93 | Q8VFT1 | Q8VFN0 | Q8VFC6 | Q8VEU5 | Q8VGB5 | Q8VFJ1 | Q8VGQ1 |
| Q9WU94 | Q8VFY4 | Q8VFQ1 | Q8VFC7 | Q8VEU6 | Q8VGC6 | Q8VFJ2 | Q8VGQ2 |
| Q9WVD7 | Q8VFY5 | Q8VFS2 | Q8VFC8 | Q8VEU8 | Q8VGC7 | Q8VFJ3 | Q8VGQ3 |
| Q9WVD8 | Q8VFZ4 | Q8VFS3 | Q8VFF8 | Q8VEU9 | Q8VGF0 | Q8VFJ4 | Q8VGQ4 |
| Q9WVD9 | Q8VFZ5 | Q8VFS4 | Q8VFF9 | Q8VEV5 | Q8VGI4 | Q8VFJ5 | Q8VGQ5 |
| Q9WVN4 | Q8VFZ6 | Q8VFS5 | Q8VFN1 | Q8VEV6 | Q8VGI5 | Q8VFJ6 | Q8VGQ6 |
| Q9WVN5 | Q8VG10 | Q8VFS6 | Q8VFT3 | Q8VEV7 | Q8VGI6 | Q8VFM8 | Q8VGQ7 |
| Q9WVN6 | Q8VG31 | Q8VFY2 | Q8VFT4 | Q8VEV8 | Q8VGR8 | Q8VG88 | Q8VGQ8 |
| Q9YH55 | Q8VG32 | Q8VFY3 | Q8VFT5 | Q8VEW2 | Q8VGR9 | Q8VGB8 | Q8VGQ9 |
| Q9P1Q5 | Q8VG98 | Q8VG14 | Q8VFU8 | Q8VEW5 | Q8VGS0 | Q8VGG3 | Q8VGR0 |
| Q9Y585 | Q8VG99 | Q8VG49 | Q8VFU9 | Q8VEW6 | Q8VGS1 | Q8VGG4 | Q8VGT4 |
| Q15619 | Q8VGC0 | Q8VG72 | Q8VFV0 | Q8VEY2 | Q8VGS2 | Q8VGG5 | Q8VGU2 |
| P34982 | Q8VGC1 | Q8VG87 | Q8VFV1 | Q8VEY3 | Q8VGS3 | Q8VGG6 | Q8VGV7 |
| | Q8VGE0 | | Q8VG12 | | Q8VGS4 | | Q8VGW7 |

What is claimed is:

1. A method of producing an olfactory GPCR, comprising:
introducing an expression cassette comprising a promoter operably linked to a nucleic acid encoding said olfactory GPCR into a macroglial cell in vitro, and
maintaining said cell under conditions suitable for production of said olfactory GPCR, to produce said olfactory GPCR.

2. The method of claim 1, wherein said macroglial cell is a myelin-producing cell.

3. The method of claim 1, wherein said macroglial cell is a Schwann, oligodendritic or olfactory ensheathing cell.

4. The method of claim 3, wherein said macroglial cell is a primary Schwann cell.

5. The method of claim 1, wherein said cell is an immortalized macroglial cell.

6. The method of claim 1, wherein said olfactory GPCR is detectable at the cell surface.

7. A method of screening for an olfactory modulator, comprising:
producing an olfactory GPCR in a macroglial cell according to the method of claim 1, wherein said olfactory GPCR is coupled to a G protein;
contacting said cell with a candidate agent; and
assessing the effect of said candidate agent on an activity of said olfactory GPCR,
wherein a candidate agent that modulates an activity of said olfactory GPCR is an olfactory modulator.

8. A method of screening for a modulator of an olfactory GPCR, comprising:
producing said olfactory GPCR in a macroglial cell according to the method of claim 1, wherein said olfactory GPCR is coupled to a G protein;
contacting said cell with a candidate agent; and
assessing the effect of said candidate agent on an activity of said olfactory GPCR,
wherein a candidate agent that modulates an activity of said olfactory GPCR is a modulator of said olfactory GPCR.

9. The method of claim 7 or claim 8, wherein said agent is a small organic molecule.

10. The method of claim 7 or claim 8, wherein said agent is an odorant.

11. The method of claim 7 or claim 8, wherein said contacting is carried out in the presence of a known agonist of the olfactory GPCR.

12. The method of claim 7 or claim 8, wherein said modulator is selected from the group consisting of agonist, partial agonist, inverse agonist, and antagonist.

13. The method of claim 7 or claim 8, wherein said assessing is through the measurement of the level of GTPγS binding.

14. The method of claim 7 or claim 8, wherein said assessing is through the measurement of the level of a second messenger selected from the group of cyclic AMP (cAMP), cyclic GMP (cGMP), inositol 1,4,5-triphosphate (IP3), diacylglycerol (DAG), and Ca2+.

15. The method of claim 14, wherein said second messenger is cAMP.

16. A method of screening for a ligand of an olfactory GPCR, comprising:
producing said olfactory GPCR in a macroglial cell according to the method of claim 1;
contacting said olfactory GPCR with a candidate agent; and
assessing the binding of said candidate agent to said olfactory GPCR.

17. The method of claim 16, wherein said candidate agent is labeled.

18. A method of screening for a ligand of an olfactory GPCR, comprising:
producing said olfactory GPCR in a macroglial cell according to the method of claim 1;
contacting said olfactory GPCR with a candidate agent in the presence of a labeled known ligand of the olfactory GPCR; and
assessing the binding of said labeled known ligand of the olfactory GPCR; wherein a decrease in binding of said labeled known ligand in the presence of said candidate agent is indicative of the candidate agent being a ligand of the olfactory GPCR.

19. A method of screening for an olfactory modulator, comprising:
producing a plurality of different olfactory GPCRs according to the method of claim 1, wherein each of said olfactory GPCRs is coupled to a G protein;
identifying a set of different olfactory GPCRs that are activated by a first agent, wherein said first agent is a known olfactory modulator;
contacting said set of GPCRs with a second agent; and
assessing an effect of said second agent on an activity of said olfactory GPCRs,
wherein a second agent that modulates one or more GPCRs of said set of different GPCRs modulated by the first agent is an olfactory modulator.

20. The method of claim 19, wherein said set comprises three or more GPCRs.

21. A macroglial cell comprising a recombinant nucleic acid encoding an olfactory GPCR.

22. A kit comprising:
a macroglial cell; and
a nucleic acid encoding an olfactory GPCR.

23. The kit of claim 22, further comprising instructions for producing said olfactory GPCR using said macroglial cell.

24. A method of identifying an odorant of interest in a sample, comprising:
contacting a sample with a plurality of macroglial cells producing a plurality of different olfactory GPCRs according to the method of claim 1, wherein each of said olfactory GPCRs is coupled to a G protein; and
assessing activation of said olfactory GPCRs for the presence of said odorant;
wherein activation of a pre-determined set of olfactory GPCRs indicates the presence of said odorant in said sample.

25. A method of determining a “fingerprint” of an odorant, comprising:
contacting a sample with a plurality of macroglial cells producing a plurality of different olfactory GPCRs according to the method of claim 1, wherein each of said olfactory GPCRs is coupled to a G protein; and
assessing activation of said olfactory GPCRs for activation by said odorant;
wherein said “fingerprint” comprises the set of different olfactory GPCRs activated by the odorant.

26. The method of claim 25, wherein said contacting is carried out in the presence of a known agonist of one or more of the plurality of olfactory GPCRs.

27. The method of any one of claims 24 to 26, wherein said plurality of cells is an addressable array of cells, wherein each address of said array contains macroglial cells producing a single recombinant olfactory GPCR.

28. The method of claim 27, wherein said GPCR activation is assessed using a light-emitting reporter of GPCR activation.


SEQUENCE LISTING (3 sequences)

SEQ ID NO: 1 - protein, 31 aa, Artificial Sequence
  synthetic peptide
MNGTEGPNFYVPFSNKTGVVYPYDVPDYAKL

SEQ ID NO: 2 - protein, 20 aa, Artificial Sequence
  synthetic peptide
MNGTEGPNFYVPFSNKTGVV

SEQ ID NO: 3 - protein, 11 aa, Artificial Sequence
  synthetic peptide
YPYDVPDYAKL